(19) United States
(12) Patent Application Publication
(10) Pub. No.: US 2010/0163038 A1
(43) Pub. Date: Jul. 1, 2010

(54) Methods and systems for presenting an inhalation experience

(75) Inventors: Roderick A. Hyde, Redmond, WA (US); Robert Langer, Newton, MA (US); Eric C. Leuthardt, St. Louis, MO (US); Robert W. Lord, Seattle, WA (US); Elizabeth A. Sweeney, Seattle, WA (US); Clarence T. Tegreene, Bellevue, WA (US); Lowell L. Wood, JR., Bellevue, WA (US)

Correspondence Address: IV - SUITER SWANTZ PC LLO, 14301 FNB PARKWAY , SUITE 220, OMAHA, NE, 68154, US

(73) Assignee: Searete LLC, a limited liability corporation of the State of Delaware

(21) Appl. No.: 12/387,321

(22) Filed: Apr. 30, 2009

Related U.S. Application Data
(63) Continuation-in-part of application No. 12/317,934, filed on Dec. 30, 2008.
Application No. 12/317,934 is a continuation-in-part of application No. 12/319,143, filed on Dec. 31, 2008.
Application No. 12/319,143 is a continuation-in-part of application No. 12/378,284, filed on Feb. 12, 2009.
Application No. 12/378,284 is a continuation-in-part of application No. 12/378,485, filed on Feb. 13, 2009.
Application No. 12/378,485 is a continuation-in-part of application No. 12/380,013, filed on Feb. 20, 2009.
Application No. 12/380,013 is a continuation-in-part of application No. 12/380,108, filed on Feb. 23, 2009.
Application No. 12/380,108 is a continuation-in-part of application No. 12/380,587, filed on Feb. 27, 2009.
Application No. 12/380,587 is a continuation-in-part of application No. 12/380,679, filed on Mar. 2, 2009.
Application No. 12/380,679 is a continuation-in-part of application No. 12/383,509, filed on Mar. 25, 2009.
Application No. 12/383,509 is a continuation-in-part of application No. 12/383,819, filed on Mar. 26, 2009.
Application No. 12/383,819 is a continuation-in-part of application No. 12/384,104, filed on Mar. 31, 2009.
Application No. 12/384,104 is a continuation-in-part of application No. 12/384,203, filed on Apr. 1, 2009.
Application No. 12/384,203 is a continuation-in-part of application No. 12/386,574, filed on Apr. 20, 2009.
Application No. 12/386,574 is a continuation-in-part of application No. 12/386,669, filed on Apr. 21, 2009.
Application No. 12/386,669 is a continuation-in-part of application No. 12/387,057, filed on Apr. 27, 2009.
Application No. 12/387,057 is a continuation-in-part of application No. 12/387,151, filed on Apr. 28, 2009.

(51) Int. Cl. A61M 16/10

(52) U.S. Cl. 128/203.14

(57) ABSTRACT

Methods, computer program products, and systems are described that include accepting an indication of a schedule for administration of an inhalation device-dispensed bioactive agent to an individual and presenting an indication of an artificial sensory experience at least partly based on the accepting an indication of the schedule for administration of the inhalation device-dispensed bioactive agent to the individual.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Dec. 30, 2008, application Ser. No. 12/317,934, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Dec. 31, 2008, application Ser. No. 12/319,143, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Feb. 12, 2009, application Ser. No. 12/378,284, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Feb. 13, 2009, application Ser. No. 12/378,485, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Feb. 20, 2009, application Ser. No. 12/380,013, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Feb. 23, 2009, application Ser. No. 12/380,108, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Feb. 27, 2009, application Ser. No. 12/380,587, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Mar. 2, 2009, application Ser. No. 12/380,679, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Mar. 25, 2009, application Ser. No. 12/383,509, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Mar. 26, 2009, application Ser. No. 12/383,819, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Mar. 31, 2009, application Ser. No. 12/384,104, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Apr. 1, 2009, application Ser. No. 12/384,203, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Apr. 20, 2009, application Ser. No. NOT YET ASSIGNED, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Apr. 21, 2009, application Ser. No. NOT YET ASSIGNED, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Apr. 27, 2009, application Ser. No. NOT YET ASSIGNED, which is currently co-pending, or is an application of which a currently co-pending application is entitled to the benefit of the filing date.
For purposes of the USPTO extra-statutory requirements, the present application constitutes a continuation-in-part of United States Patent Application entitled METHODS AND SYSTEMS FOR PRESENTING AN INHALATION EXPERIENCE, naming RODERICK A. HYDE; ROBERT LANGER; ERIC C. LEUTHARDT; ROBERT W. LORD; ELIZABETH A. SWEENEY; CLARENCE T. TEGREENE; AND LOWELL L. WOOD as inventors, filed Apr. 28, 2009, application Ser. No. NOT YET ASSIGNED, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

This description relates to methods and systems for an inhaled bioactive agent combined with an artificial sensory experience.

### SUMMARY

In one aspect, a method includes but is not limited to accepting an indication of a schedule for administration of an inhalation device-dispensed bioactive agent to an individual and presenting an indication of an artificial sensory experience at least partly based on the accepting an indication of the schedule for administration of the inhalation device-dispensed bioactive agent to the individual. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a method includes but is not limited to accepting an indication of at least one of a pharmacokinetic or pharmacodynamic profile of an inhalation device-dispensed bioactive agent and an indication of a schedule for administration of the inhalation device-dispensed bioactive agent to an individual and presenting an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a method includes but is not limited to accepting an indication of an inhalation device-dispensed bioactive agent administration for an individual, determining at least one of a pharmacokinetic or pharmacodynamic profile of the inhalation device-dispensed bioactive agent and presenting an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a method includes but is not limited to accepting an indication of at least one inhalation device-dispensed bioactive agent and an indication of a schedule for administration of the inhalation device-dispensed bioactive agent to an individual and presenting an indication of an artificial sensory experience at least partly based on at least one of a pharmacokinetic profile or pharmacodynamic profile of the inhalation device-dispensed bioactive agent and the indication of the schedule for administration of the inhalation device-dispensed bioactive agent to the individual. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to means for accepting an indication of a schedule for administration of an inhalation device-dispensed bioactive agent to an individual and means for presenting an indication of an artificial sensory experience at least partly based on the accepting an indication of the schedule for administration of the inhalation device-dispensed bioactive agent to the individual. In addition to the foregoing, other method aspects are described in the claims, drawings, and text forming a part of the present disclosure.

In one aspect, a system includes but is not limited to circuitry for accepting an indication of a schedule for administration of an inhalation device-dispensed bioactive agent to an individual and circuitry for presenting an indication of an artificial sensory experience at least partly based on the accepting an indication of the schedule for administration of the inhalation device-dispensed bioactive agent to the individual. In addition to the foregoing, other method aspects are described in the claims, drawings, and text forming a part of the present disclosure.

In one aspect, a computer program product includes but is not limited to a signal-bearing medium bearing one or more instructions for accepting an indication of a schedule for administration of an inhalation device-dispensed bioactive agent to an individual and one or more instructions for presenting an indication of an artificial sensory experience at least partly based on the accepting an indication of the schedule for administration of the inhalation device-dispensed bioactive agent to the individual. In addition to the foregoing, other method aspects are described in the claims, drawings, and text forming a part of the present disclosure.

In one aspect, a system includes but is not limited to a computing device and instructions that when executed on the computing device cause the computing device to accept an indication of a schedule for administration of an inhalation device-dispensed bioactive agent to an individual and present an indication of an artificial sensory experience at least partly based on the accepting an indication of the schedule for administration of the inhalation device-dispensed bioactive agent to the individual. In addition to the foregoing, other method aspects are described in the claims, drawings, and text forming a part of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 illustrates an exemplary environment in which one or more technologies may be implemented.

FIG. 2 illustrates an exemplary environment in which one or more technologies may be implemented.

FIG. 3 illustrates an exemplary inhalation device.

FIG. 4 illustrates an exemplary environment in which one or more technologies may be implemented.

FIG. 5 illustrates an exemplary environment in which one or more technologies may be implemented.

FIG. 6 illustrates an operational flow representing example operations related to combining an inhaled bioactive agent and an artificial sensory experience.

FIG. 7 illustrates an alternative embodiment of the operational flow of FIG. 6.

FIG. 8 illustrates an alternative embodiment of the operational flow of FIG. 6.

FIG. 9 illustrates an alternative embodiment of the operational flow of FIG. 6.

FIG. 10 illustrates an alternative embodiment of the operational flow of FIG. 6.

FIG. 11 illustrates an alternative embodiment of the operational flow of FIG. 6.

FIG. 12 illustrates an alternative embodiment of the operational flow of FIG. 6.

FIG. 13 illustrates an alternative embodiment of the operational flow of FIG. 6.

FIG. 14 illustrates an alternative embodiment of the operational flow of FIG. 6.

FIG. 15 illustrates an alternative embodiment of the operational flow of FIG. 6.

FIG. 16 illustrates a computer program product related to combining an inhaled bioactive agent and an artificial sensory experience.

FIG. 17 illustrates a system related to combining an inhaled bioactive agent and an artificial sensory experience.

FIG. 18 illustrates an exemplary environment in which one or more technologies may be implemented.

FIG. 19 illustrates an exemplary environment in which one or more technologies may be implemented.

FIG. 20 illustrates an exemplary environment in which one or more technologies may be implemented.

FIG. 21 illustrates an exemplary environment in which one or more technologies may be implemented.

FIG. 22 illustrates an operational flow representing example operations related to combining an inhaled bioactive agent and an artificial sensory experience.

FIG. 23 illustrates an alternative embodiment of the operational flow of FIG. 22.

FIG. 24 illustrates an alternative embodiment of the operational flow of FIG. 22.

FIG. 25 illustrates an alternative embodiment of the operational flow of FIG. 22.

FIG. 26 illustrates an alternative embodiment of the operational flow of FIG. 22.

FIG. 27 illustrates an alternative embodiment of the operational flow of FIG. 22.

FIG. 28 illustrates an alternative embodiment of the operational flow of FIG. 22.

FIG. 29 illustrates an alternative embodiment of the operational flow of FIG. 22.

FIG. 30 illustrates an alternative embodiment of the operational flow of FIG. 22.

FIG. 31 illustrates an alternative embodiment of the operational flow of FIG. 22.

FIG. 32 illustrates an alternative embodiment of the operational flow of FIG. 22.

FIG. 33 illustrates an alternative embodiment of the operational flow of FIG. 22.

FIG. 34 illustrates an alternative embodiment of the operational flow of FIG. 22.

FIG. 35 illustrates an alternative embodiment of the operational flow of FIG. 22.

FIG. 36 illustrates an alternative embodiment of the operational flow of FIG. 22.

FIG. 37 illustrates an alternative embodiment of the operational flow of FIG. 22.

FIG. 38 illustrates an alternative embodiment of the operational flow of FIG. 22.

FIG. 39 illustrates an alternative embodiment of the operational flow of FIG. 22.

FIG. 40 illustrates a computer program product related to combining an inhaled bioactive agent and an artificial sensory experience.

FIG. 41 illustrates a system related to combining an inhaled bioactive agent and an artificial sensory experience.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

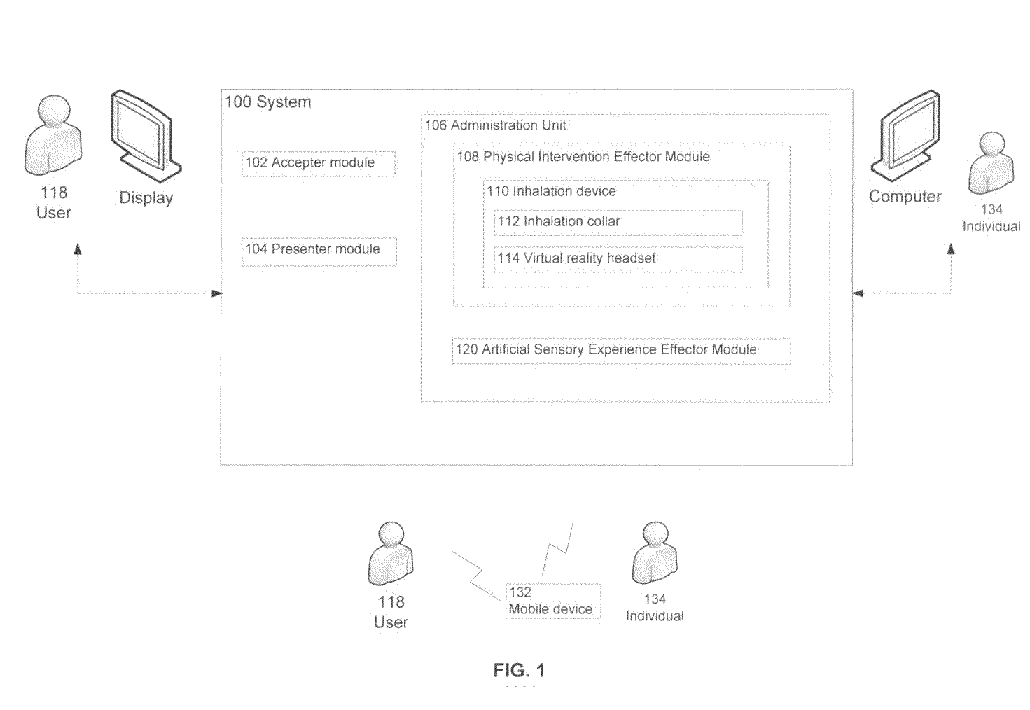

FIG. 1 illustrates system 100 for accepting an indication of at least one health-related condition and/or presenting an indication of at least one artificial sensory experience and an indication of at least one inhalation therapy at least partially based on the accepting at least one indication of a health-related condition. The system 100 may include accepter module 102, presenter module 104, and/or administration unit 106. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, system 3200 may include mobile device 132.

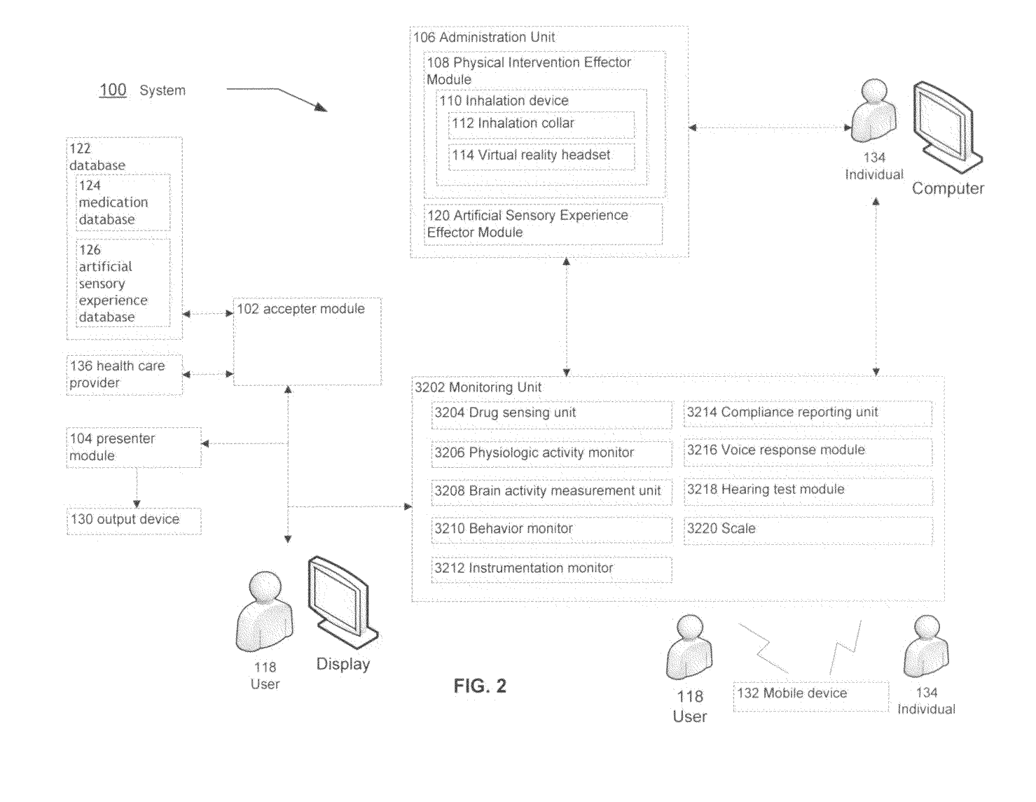

FIG. 2 illustrates system 100 for accepting an indication of at least one health-related condition and/or presenting an indication of at least one artificial sensory experience and an indication of at least one inhalation therapy at least partially based on the accepting at least one indication of a health-related condition. The system 100 may include accepter module 102, presenter module 104, administration unit 106, and/or monitoring unit 3202. Accepter module 102 may receive and/or transmit information and/or data to and/or from user 118, database 122, presenter module 3410, output device 130, and/or health care provider 136. Database 122 may include medication database 124 and/or artificial sensory experience database 126. Monitoring unit 3202 may monitor individual 134 and may include drug sensing unit 3204, physiologic activity monitor 3206, brain activity measurement unit 3208, behavior monitor 3210, instrumentation monitor 3212, compliance reporting unit 3214, voice response module 3216, hearing test module 3218, and/or scale 3220. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, mobile device 132 may communicate with accepter module 102, presenter module 104, healthcare provider 136, user 118, individual 134, monitoring unit 3202, and/or administration unit 3222.

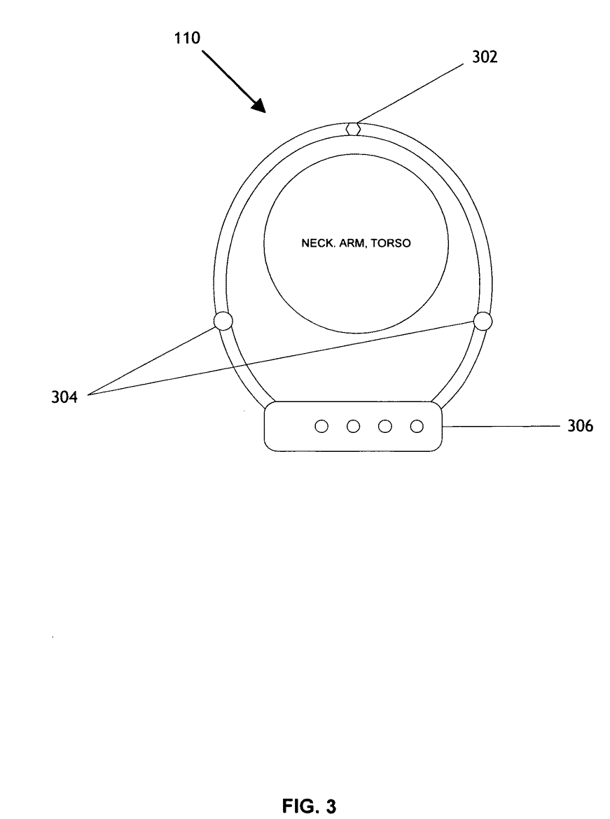

FIG. 3 illustrates an exemplary inhalation device 110. An exemplary inhalation device 110 may include a closure device, a transducer, and/or a dispensing reservoir. Inhalation device 110 may include, for example, a collar, a necklace, and/or a bracelet. Inhalation device 110 may include tubing, a chain, a polymer, a metal, a textile, and may be solid and/or hollow. Closure device 302 may include a buckle, Velcro, a snap, a clasp, a lock, a coupler, elastic, and/or magnets. Transducer 304 may include a blood glucose monitor, a blood oxygen monitor, means for sending a signal to a reservoir to dispense medication, such as an antenna, means for powering the unit, such as a battery, memory, and/or a computer processor. Dispensing reservoir 306 may include means for power, such as a battery, means for receiving conditional input, such as a processor and/or memory, means for dispensing a bioactive agent in aerosol, dust and/or vapor form, such as a nebulizer, a sprayer, and/or a nozzle. Additionally, the dispensing reservoir 306 may be removable and/or refillable.

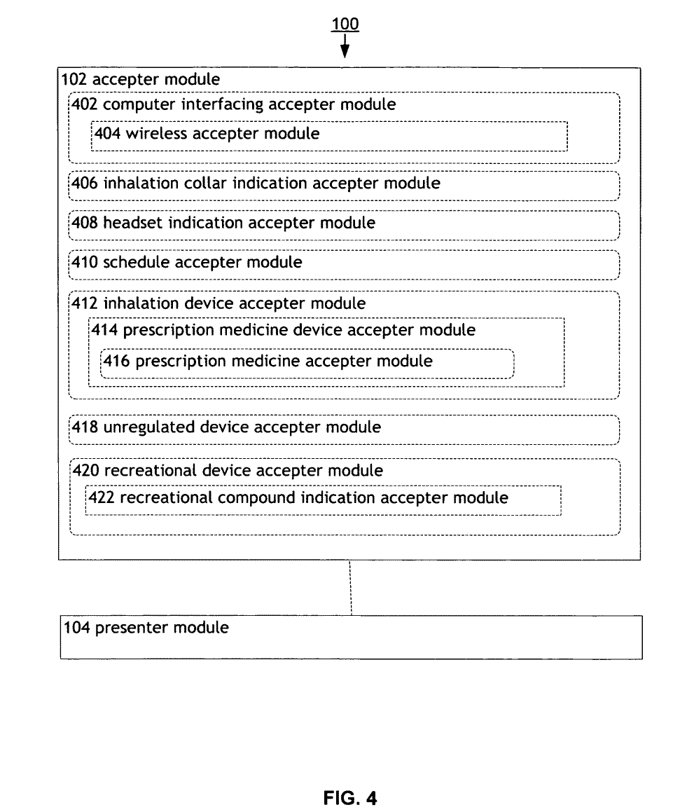

FIG. 4 further illustrates system 100 including accepter module 102 and/or presenter module 104. Accepter module 102 may include computer interfacing accepter module 402, inhalation collar indication accepter module 406, headset indication accepter module 408, schedule accepter module 410, inhalation device accepter module 412, unregulated device accepter module 418, and/or recreational device accepter module 420. Computer interfacing accepter module 402 may include wireless accepter module 404. Inhalation device accepter module 412 may include prescription medicine device accepter module 414 and/or prescription medicine accepter module 416. Recreational device accepter module 420 may include recreational compound indication accepter module 422.

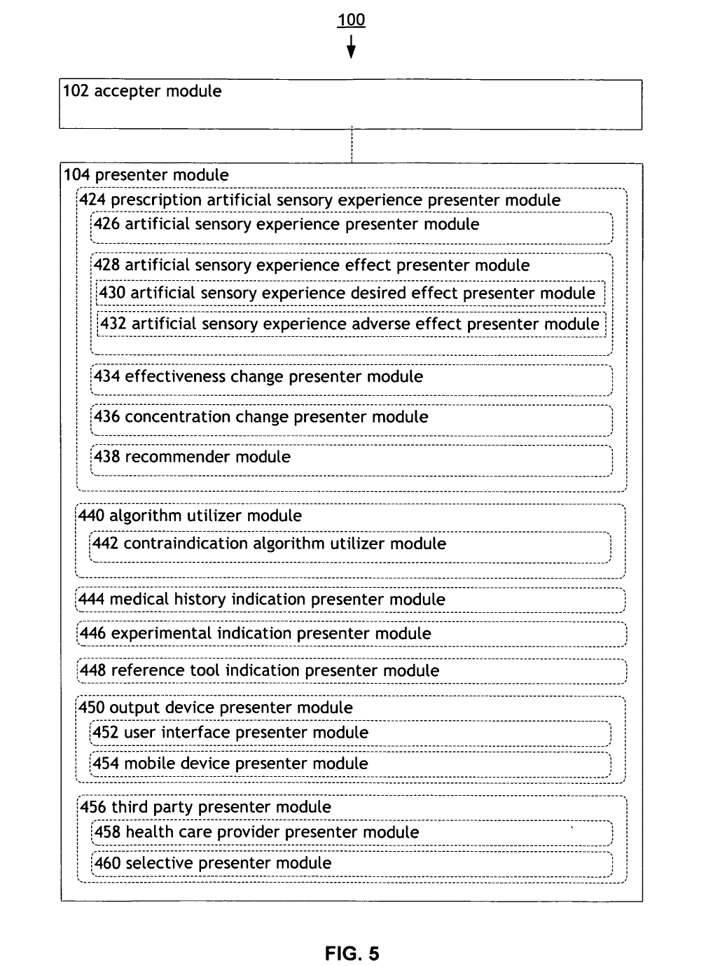

FIG. 5 illustrates system 100 including accepter module 102 and/or presenter module 104. Presenter module 104 may include prescription artificial sensory experience presenter module 424, algorithm utilizer module 440, medical history indication presenter module 444, experimental indication presenter module 446, reference tool indication presenter module 448, output device presenter module 450, and/or third party presenter module 456. Prescription artificial sensory experience presenter module 424 may include artificial sensory experience presenter module 426, artificial sensory experience effect presenter module 428, effectiveness change presenter module 434, concentration change presenter module 436, and/or recommender module 438. Artificial sensory experience effect presenter module 428 may include artificial sensory experience desired effect presenter module 430 and/or artificial sensory experience adverse effect presenter module 432. Algorithm utilizer module 440 may include contraindication algorithm utilizer module 442. Output device presenter module 450 may include user interface presenter module 452 and/or mobile device presenter module 454. Third party presenter module 456 may include health care provider presenter module 458 and/or selective presenter module 460.

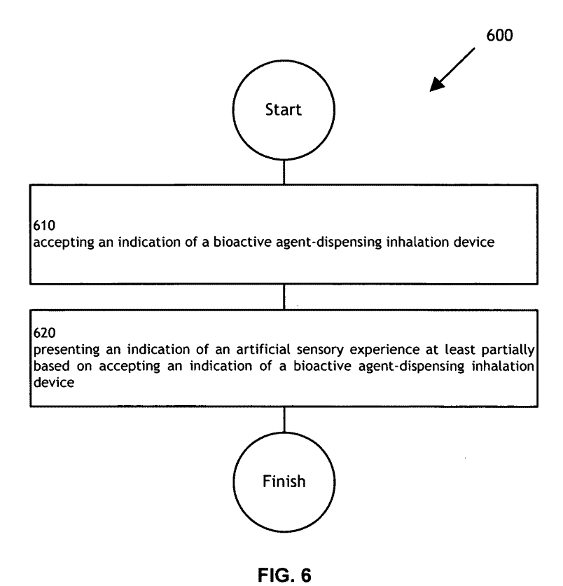

FIG. 6 illustrates an operational flow 600 representing example operations related to accepting an indication of at least one health-related condition and presenting an indication of at least one artificial sensory experience and an indication of at least one inhalation therapy at least partially based on the accepting at least one indication of a health-related condition. In FIG. 6 and in following figures that include various examples of operational flows, discussion and explanation may be provided with respect to the above-described examples of FIGS. 1 through 5, and/or with respect to other examples and contexts. However, it should be understood that the operational flows may be executed in a number of other environments and contexts, and/or in modified versions of FIGS. 1 through 5. Also, although the various operational flows are presented in the sequence(s) illustrated, it should be understood that the various operations may be performed in other orders than those which are illustrated, or may be performed concurrently.

After a start operation, the operational flow 600 moves to operation 610. Operation 610 depicts accepting an indication of at least one health-related condition. For example, as shown in FIGS. 1 through 5, accepter module 102 may accept an indication of a bioactive agent-dispensing inhalation device. One example of a bioactive agent-dispensing inhalation device may include an inhaler used for delivering a bioactive agent into the body using a body airway. Some other examples may include a collar, necklace, and/or a bracelet with a bioactive agent dispenser proximate to the nose, mouth, and/or inhalation route. In one embodiment, accepter module 102 may accept an indication of a bioactive agent-dispensing collar for dispensing a medication, such as a steroid and/or a bronchodilator. In some instances, accepter module 102 may include a computer processor, a user interface, and/or computer memory.

Then, operation 620 depicts presenting an indication of at least one artificial sensory experience and an indication of at least one inhalation therapy at least partially based on the accepting at least one indication of a health-related condition. For example, as shown in FIGS. 1 through 5, presenter module 104 may present an indication of a virtual world at least partially based on accepting an indication of a bioactive agent-dispensing inhalation device. One example of an artificial sensory experience may include a virtual world and/or other computer-simulated experience. Other examples of an artificial sensory experience may include experiences triggering sight, smell, hearing, touch, and/or taste. For example, presenter module 104 may present an indication of an artificial sensory experience including a virtual scent environment, which may include olfactory stimulation for improving memory. In an additional embodiment, presenter module 104 may present an indication of an artificial sensory experience including a virtual experience where the user is exposed to a virtual mountain environment coupled with a bronchodilator dose from a bioactive agent-dispensing inhalation collar. In this embodiment, the combination bronchodilator and virtual world treatment may serve to help an asthma sufferer to learn effective breathing techniques. Presenting an indication of an artificial sensory experience may include presenting the indication to a physician, to a computer monitor, to a mobile device, and/or to a third party. In some instances, presenter module 104 may include a computer processor and/or a communication device, such as a printer, a computer monitor, and/or a speaker.

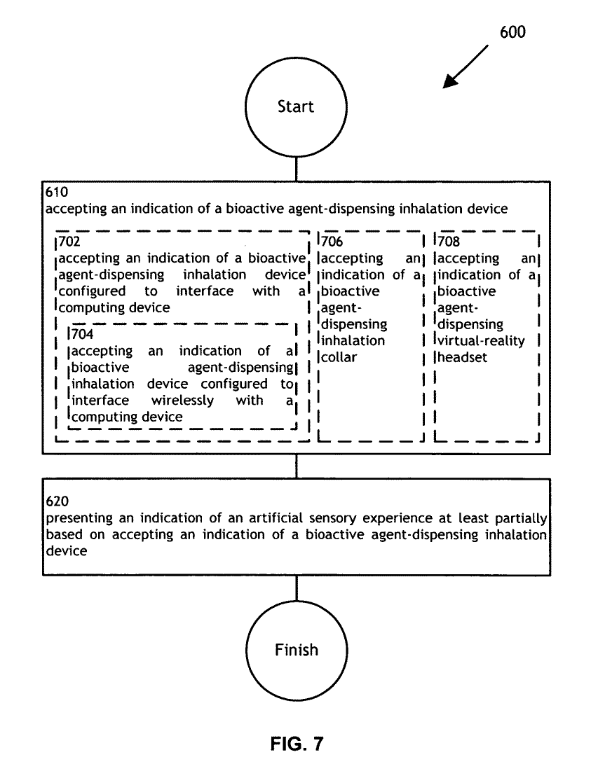

FIG. 7 illustrates alternative embodiments of the example operational flow 600 of FIG. 6. FIG. 7 illustrates example embodiments where operation 610 may include at least one additional operation. Additional operations may include operation 702, operation 704, operation 706, and/or operation 708.

Operation 702 illustrates accepting an indication of a health-related physical condition. For example, as shown in FIGS. 1 through 5, computer interfacing accepter module 402 may accept an indication of a bioactive agent-dispensing inhalation device configured to interface with a computing device. In one embodiment, computer interfacing accepter module 402 may accept an indication of a bioactive agent-dispensing inhalation device configured to interface with a virtual game, such as World of Warcraft. Some examples of a computing device may include a personal computer, a virtual-reality helmet and/or headset, and/or a virtual environment. In some instances, computer interfacing accepter module 402 may include a computer processor.

Further, operation 704 illustrates accepting an indication of a bioactive agent-dispensing inhalation device configured to interface wirelessly with a computing device. For example, as shown in FIGS. 1 through 5, wireless accepter module 404 may accept an indication of a bioactive agent-dispensing inhalation device configured to interface wirelessly with a computing device. In one embodiment, wireless accepter module 404 may accept an indication of a wireless inhalation collar configured to interface wirelessly with a computer coupled to wireless video glasses. In this embodiment, both the inhalation collar and the video glasses may be wirelessly connected to the computer. The wireless bioactive agent-dispensing inhalation device may be wirelessly coupled to a computing device using, for example, an IEEE 802.11 computer network and/or a Bluetooth wireless sensor network. One example of wireless video glasses may include Qingbar GP300 video glasses available from 22moo International Pty Ldt., Cabramatta NSW, Australia. In some instances, wireless accepter module 404 may include a computer processor and/or a wireless receiving device, such as a receiving antenna.

Operation 706 illustrates accepting an indication a health-related condition from a medical history. For example, as shown in FIGS. 1 through 5, inhalation collar indication accepter module 406 may accept an indication of a bioactive agent-dispensing inhalation collar. A bioactive agent-dispensing inhalation collar may include a collar with, for example, means for dispensing a bioactive agent, such as a reservoir and/or an accompanying valve and spray nozzle. Additionally, means for dispensing a bioactive agent may include means for dispensing an aerosol, vapor, a powder (e.g. pulmicort and/or foradil), and/or a mist, such as a nebulizer, means for measuring and/or detecting a condition, such as blood oxygen level and/or body temperature, and/or means for processing information, such as a computer processor and/or computer memory. Further, a bioactive agent may be dispensed and/or dispersed in and/or include a surfactant. In one embodiment, inhalation collar indication accepter module 406 may accept an indication of a bioactive agent-dispensing collar having means for dispensing a steroid as an aerosol. Further, a bioactive agent-dispensing inhalation collar may include means for power, such as a battery and/or circuitry for receiving power from an external source, such as an AC adapter power supply. In some instances, inhalation collar indication accepter module 406 may include a computer processor.

Operation 708 illustrates accepting an indication of a bioactive agent-dispensing virtual-reality headset. For example, as shown in FIGS. 1 through 5, headset indication accepter module 408 may accept an indication of a bioactive agent-dispensing virtual-reality headset. A virtual-reality headset may include a microphone, headphones or speakers for hearing, and/or a display. A virtual-reality headset may be configured for enabling a user to engage in an artificial sensory experience including sound, smell, and/or sight. One example of a virtual-reality headset may include a virtual reality helmet configured to give the user a 360° view of a mountain landscape while dispensing a bronchodilator for helping the user learn improved breathing techniques. Another example of a virtual reality head set may include an Olympus Eye-Trek FMD-200-TFT active matrix head mounted display with Speaker, available from Olympus America Inc., Center Valley Pa. In some instances, headset indication accepter module 408 may include a computer processor.

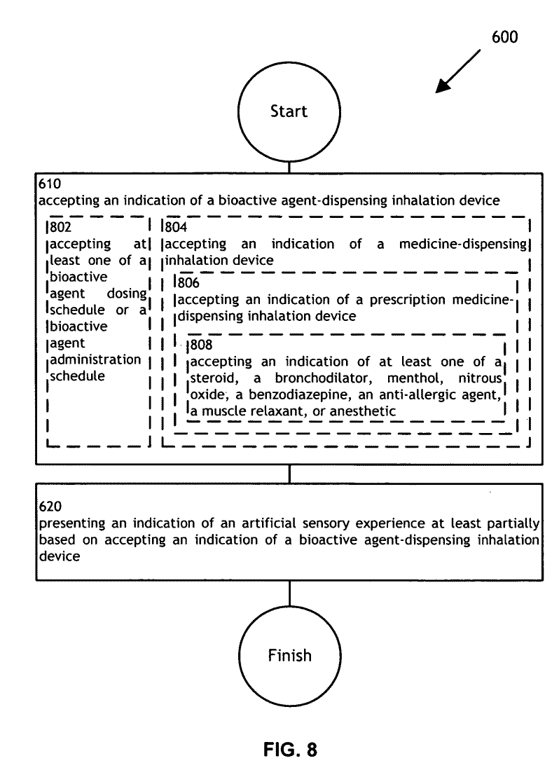

FIG. 8 illustrates alternative embodiments of the example operational flow 600 of FIG. 6. FIG. 8 illustrates example embodiments where the operation 610 may include at least one additional operation. Additional operations may include an operation 802, an operation 804, an operation 806, and/or an operation 808.

Operation 802 illustrates accepting an indication of a health-related mental condition. For example, as shown in FIGS. 1 through 5, schedule accepter module 410 may accept at least one of a bioactive agent dosing schedule or a bioactive agent administration schedule. Accepting a bioactive agent dosing schedule or a bioactive agent administration schedule may include accepting from a computer processor, accepting from a memory device, and/or accepting from a user input. In one embodiment, schedule accepter module 410 may accept a dosing schedule specifying a bronchodilator administration dosage for a specified time period, such one dose from an inhalation device once every thirty minutes. In another embodiment, schedule accepter module 410 may accept a bioactive agent administration schedule specifying at least one time a bronchodilator may be administered. In some instances, schedule accepter module 410 may include a computer processor.

Operation 804 illustrates accepting an indication of a medicine-dispensing inhalation device. For example, as shown in FIGS. 1 through 5, inhalation device accepter module 412 may accept an indication of a medicine-dispensing inhalation device. A medicine-dispensing inhalation device may include a device for dispensing a substance for treating a disease and/or illness. For example, a medicine-dispensing inhalation device may include an inhaler as described in Robertson et al., U.S. Pat. No. 7,383,837, which is incorporated herein by reference. Some other examples may include a metered-dose inhaler, a dry powder inhaler, and/or a nebulizer. In one embodiment, inhalation device accepter module 412 may accept an indication of a medicine-dispensing metered-dose inhaler configured to dispense albuterol. In some instances, inhalation device accepter module 412 may include a computer processor.

Further, operation 806 illustrates accepting an indication of a health-related condition from a user input. For example, as shown in FIGS. 1 through 5, prescription medicine device accepter module 414 may accept an indication of a prescription medicine-dispensing inhalation device. A prescription medicine-dispensing inhalation device may include a device configured to dispense a medication only available from a licensed health care provider. Some examples of a prescription medication available from a licensed health care provider may include albuterol, coricosteroids, nitrous oxide, a benzodiazepine, Theophylline, nedocromil sodium, and/or fluticasone/salmeterol. In one embodiment, prescription medicine device accepter module 414 may accept an indication of a prescription medicine-dispensing inhalation device configured for dispensing ciclesonide. In some instances, prescription medicine device accepter module 414 may include a computer processor.

Further, operation 808 illustrates indication of at least one of a prescribed artificial sensory experience or a prescribed inhalation therapy. For example, as shown in FIGS. 1 through 5, prescription medicine accepter module 416 may accept an indication of at least one of a steroid, a bronchodilator, menthol, nitrous oxide, a benzodiazepine, or halothane. One example of a steroid may include an anabolic steroid, which may be a derivative of androgens (such as testosterone), for stimulating growth. Another example of a steroid may include a corticosteroid, which may be often used as an anti-inflammatory prescribed for asthma. A bronchodilator may include a substance that dilates the bronchi and bronchioles decreasing airway resistance and thereby facilitating airflow. Menthol may include an organic and/or synthetic compound with local anesthetic and counterirritant qualities often used for relieving throat irritation and/or as a decongestant. Nitrous oxide may include a gas often used as a weak general anesthetic. A benzodiazepine may include a class of psychoactive drugs with varying hypnotic, sedative, anxiolytic, anticonvulsant, muscle relaxant and amnesic properties, which may be mediated by slowing down the central nervous system. In one embodiment, prescription medicine accepter module 416 may accept an indication of a benzodiazepine. One example of benzodiazepine delivery through an inhalation route may be disclosed in Kim et al., U.S. Patent Publication No. 2003/0032638, which is incorporated herein by reference. An anti-allergic agent may include an agent configured to block the action of allergic mediators and/or to prevent activation of cells and degranulation processes. Some examples of an anti-allergic agent may include an antihistamine and/or cromones like mast cell stabilizers, such as cromoglicic acid and nedocromil sodium. A muscle relaxant may include a bioactive agent for affecting skeletal muscle function and/or decreasing muscle tone. One example of a skeletal muscle relaxant may include carisoprodol. Additionally, a muscle relaxant may include a smooth muscle relaxant. One example of a smooth muscle relaxant may include a methylxanthine, such as Theophylline. An anesthetic may include an inhalational general anesthetic, such as halothane, desflurane, enflurane, isoflurane, and/or sevoflurane. In some instances, prescription medicine accepter module 416 may include a computer processor.

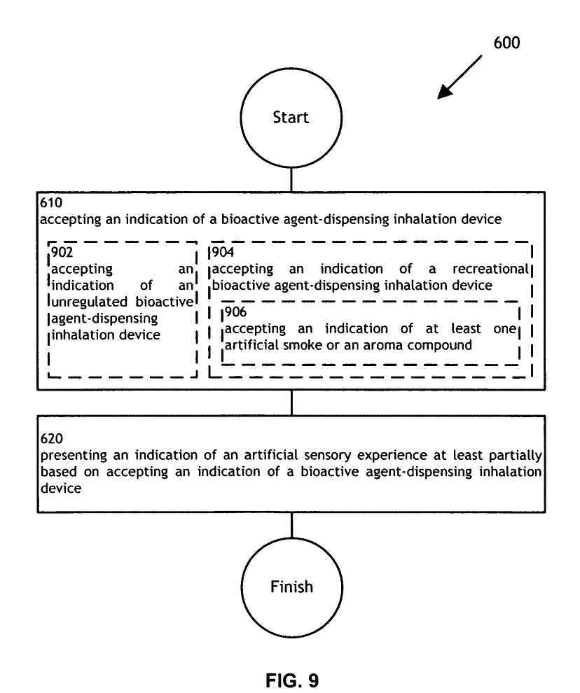

FIG. 9 illustrates alternative embodiments of the example operational flow 600 of FIG. 6. FIG. 9 illustrates example embodiments where the operation 610 may include at least one additional operation. Additional operations may include an operation 902, an operation 904, and/or an operation 906.

Operation 902 illustrates accepting an indication of an unregulated bioactive agent-dispensing inhalation device. For example, as shown in FIGS. 1 through 5, unregulated device accepter module 418 may accept an indication of an unregulated bioactive agent-dispensing inhalation device. In one embodiment, unregulated device accepter module 418 may accept an indication of an oxygen-dispensing inhalation device. Some examples of an unregulated bioactive agent may include oxygen, aromas used for aromatherapy, and/or menthol. In another embodiment, unregulated device accepter module 418 may accept an indication of an aromatherapeutic-dispensing inhalation collar. In some instances, unregulated device accepter module 418 may include a computer processor.

Operation 904 illustrates accepting an indication of a recreational bioactive agent-dispensing inhalation device. For example, as shown in FIGS. 1 through 5, recreational device accepter module 420 may accept an indication of a recreational bioactive agent-dispensing inhalation device. In one embodiment, recreational device accepter module 420 may accept an indication of a recreational bioactive agent-dispensing inhalation device. Some examples of a recreational bioactive agent may include an aroma compound used for aromatherapy and/or artificial smoke. Other examples of a recreational bioactive agent may include incense and/or smoke, such as incense and/or smoke used in a religious rite. In some instances, recreational device accepter module 420 may include a computer processor.

Further, operation 906 illustrates accepting an indication of at least one artificial smoke or an aroma compound. For example, as shown in FIGS. 1 through 5, recreational compound indication accepter module 422 may accept an indication of at least one artificial smoke or an aroma compound. In one embodiment, recreational compound indication accepter module 422 may accept an indication of artificial smoke while experiencing a virtual world. In another embodiment, recreational compound indication accepter module 422 may accept an indication of lemon oil while experiencing an artificial sensory experience. In this embodiment, the use of lemon oil as an aromatherapeutic may serve to enhance a user's mood and/or provide relaxation. In some instances, recreational compound indication accepter module 422 may include a computer processor.

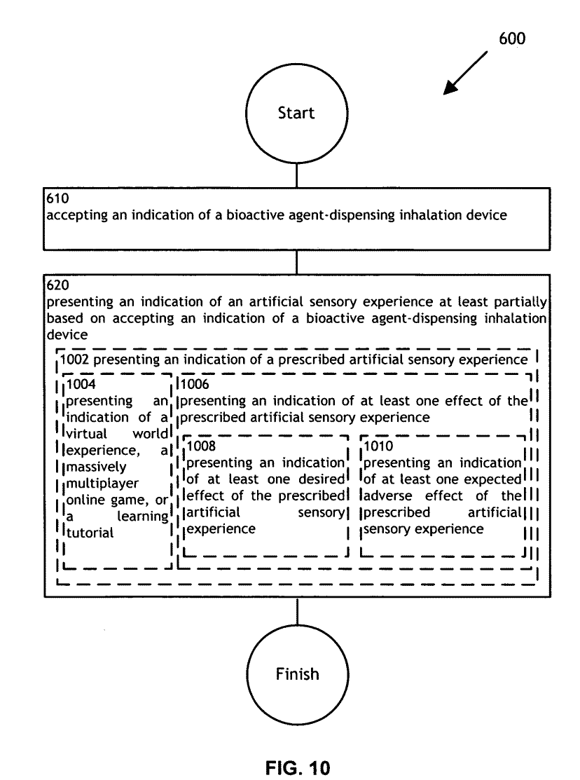

FIG. 10 illustrates alternative embodiments of the example operational flow 600 of FIG. 6. FIG. 10 illustrates example embodiments where operation 620 may include at least one additional operation. Additional operations may include operation 1002, operation 1004, operation 1006, operation 1008, and/or operation 1010.

Operation 1002 illustrates indication of at least one of a prescribed artificial sensory experience or a prescribed inhalation therapy. For example, as shown in FIGS. 1 through 5, prescription artificial sensory experience presenter module 424 may present an indication of a prescribed artificial sensory experience. A prescribed artificial sensory experience may include any artificial sensory experience prescribed by a health care professional, such as a physician, a mental health specialist, a nurse, a physical therapist, an occupational therapist, a chiropractor, and/or a homeopathic practitioner. In one embodiment, prescription artificial sensory experience presenter module 424 may present an indication of a virtual world prescribed by a psychiatrist. In this embodiment, the prescribed virtual world may be configured to be administered in conjunction with a prescribed bioactive agent. Administering a prescribed bioactive agent in conjunction with a prescribed artificial sensory experience may serve to increase efficacy of the combined therapy, for example, by serving as a distraction from pain. In some instances, prescription artificial sensory experience presenter module 424 may include a computer processor and/or a display device, such as a computer monitor and/or a printer.

Further, operation 1004 illustrates an indication of at least one of a virtual world experience, a massively multiplayer online game, or a learning tutorial. For example, as shown in FIGS. 1 through 5, artificial sensory experience presenter module 426 may present an indication of a virtual world experience, a massively multiplayer online game, or a learning tutorial. A virtual world experience may include a computer-based simulated environment intended to be interactive. Some examples of a virtual world experience may include a text-based chat room, computer conferencing, an online game, a single player game, and/or a computer tutorial. A massively multiplayer online game may include a video game capable of supporting multiple players, such as World of Warcraft and/or SecondLife. Additionally, a massively multiplayer online game may include an experience, such as a game, which may include a video game or other interactive experience involving numbers of individuals, for example, a religious ceremony or combat training exercise. An online learning tutorial may include a screen recording, a written document (either online or downloadable), or an audio file, where a user may be given step by step instructions on how to do something. In one embodiment, artificial sensory experience presenter module 426 may present an indication of a virtual world experience, such as World of Warcraft. In some instances, artificial sensory experience presenter module 426 may include a computer processor.

Further, operation 1006 illustrates indication of at least one effect of the indication of at least one of a prescribed artificial sensory experience. For example, as shown in FIGS. 1 through 5, artificial sensory experience effect presenter module 428 may present an indication of at least one effect of the prescribed artificial sensory experience. In one embodiment, artificial sensory experience effect presenter module 428 may present an indication of at least one effect of the prescribed artificial sensory experience. An effect may include a reaction and/or thing that occurs as a result of the artificial sensory experience. For example, an effect may include a side effect, a desired effect, and/or an adverse effect. Some examples of an effect may include an increased bioactive agent efficacy, dizziness, and/or a decreased heart rate. In some instances, artificial sensory experience effect presenter module 428 may include a computer processor.

Further, operation 1008 illustrates presenting an indication of at least one expected desired effect of the prescribed artificial sensory experience. For example, as shown in FIGS. 1 through 5, artificial sensory experience desired effect presenter module 430 may present an indication of at least one desired effect of the prescribed artificial sensory experience. Some examples of a desired effect may include effects such as an increased bioactive agent efficacy, a cured illness and/or condition, and/or a changed behavior. In one embodiment, artificial sensory experience desired effect presenter module 430 may present an indication of an increased opioid efficacy measured by self pain evaluation by an individual. In some instances, artificial sensory experience desired effect presenter module 430 may include a computer processor and/or a display, such as a monitor and/or a printer.

Further, operation 1010 illustrates an indication of at least one prescribed inhalation therapy. For example, as shown in FIGS. 1 through 5, artificial sensory experience adverse effect presenter module 432 may present an indication of an expected adverse effect of the prescribed artificial sensory experience. An adverse effect may include a harmful and/or undesired effect resulting from an intervention, such as an artificial sensory experience. Some examples of an adverse effect may include headache, dizziness, depression, bleeding, seizure, and/or fever. In one embodiment, artificial sensory experience adverse effect presenter module 432 may present an indication of fever in an individual while being administered a prescribed artificial sensory experience and bioactive agent. In some instances, artificial sensory experience adverse effect presenter module 432 may include a computer processor, a display device, such as a monitor and/or printer, and/or medical instrumentation, such as a thermometer configured for measuring a body temperature.

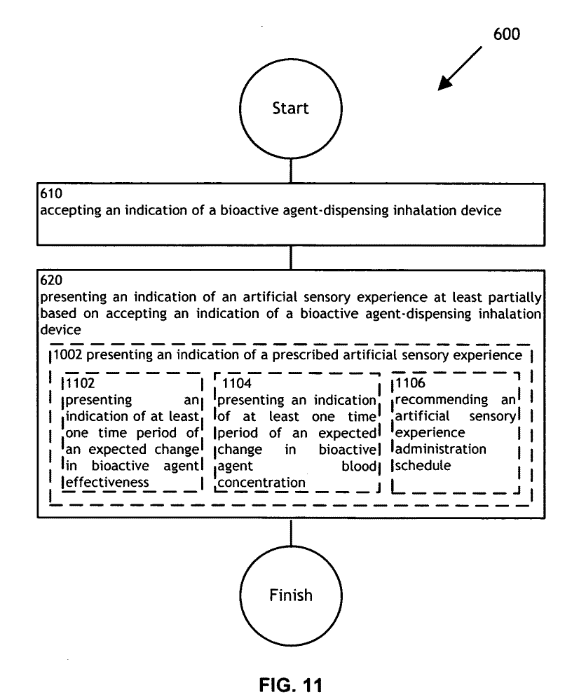

FIG. 11 illustrates alternative embodiments of the example operational flow 600 of FIG. 6. FIG. 11 illustrates example embodiments where operation 620 may include at least one additional operation. Additional operations may include operation 1102, operation 1104, and/or operation 1106.

Operation 1102 illustrates an indication of at least one prescribed bioactive agent. For example, as shown in FIGS. 1 through 5, effectiveness change presenter module 434 may present an indication of at least one time period of an expected change in bioactive agent effectiveness. In one embodiment, effectiveness change presenter module 434 may present an indication of a time period when an opioid is expected to decrease in effectiveness. Such an indication of decrease and/or change in bioactive agent effectiveness may serve to indicate an appropriate time period for administering and/or modifying an artificial sensory experience to compensate for a change in bioactive agent efficacy. In another embodiment, effectiveness change presenter module 434 may present an indication of a time period where a blood stream morphine concentration drops. This time period of low blood stream morphine concentration may be appropriate for presenting an immersive virtual world for serving as a distraction to any increase in pain caused by lowered morphine concentration. In some instances, effectiveness change presenter module 434 may include a computer processor.

Further, operation 1104 illustrates an indication of at least one time period of an expected change in bioactive agent blood concentration. For example, as shown in FIGS. 1 through 5, concentration change presenter module 436 may present an indication of at least one time period of an expected change in bioactive agent blood concentration. In one embodiment, concentration change presenter module 436 may present an indication of a one hour time period of an expected change in hydrocodone blood concentration. Indicating a time period of a change in blood concentration may serve to help determine an artificial sensory experience administration schedule. For example, if a bioactive agent blood concentration is expected to be reduced during a certain time period, an artificial sensory experience configured for distracting an individual from pain may be selected for administration during that time period. In some instances, concentration change presenter module 436 may include a computer processor and/or a display device, such as a printer and/or a computer monitor.

Further, operation 1106 illustrates recommending at least one of an artificial sensory experience administration schedule. For example, as shown in FIGS. 1 through 5, recommender module 438 may recommend an artificial sensory experience administration schedule. In one embodiment, recommender module 438 may recommend a time schedule for administration of a virtual world experience. A time schedule may be recommended by taking into account factors involving the individual and/or the bioactive agent. For example, efficacy of the bioactive agent versus time may be a factor, such as a time period when the bioactive agent is less effective. Efficacy of the bioactive agent may be a factor in determining when an artificial sensory experience is administered because of the potential for the artificial sensory experience to compensate for a changed bioactive agent efficacy. An additional factor may include an attribute of the individual, such as how a bioactive agent and/or specific artificial sensory experience affects the individual, for example a side effect. Another example of recommending an artificial sensory experience may be found in Akazawa et al., U.S. Pat. No. 7,155,680, which is incorporated herein by reference. In some instances, recommender module 438 may include a computer processor.

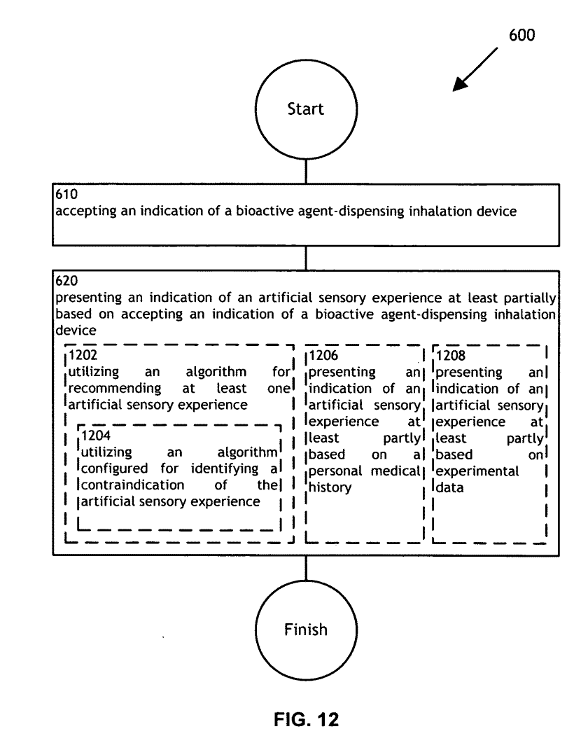

FIG. 12 illustrates alternative embodiments of the example operational flow 600 of FIG. 6. FIG. 12 illustrates example embodiments where operation 620 may include at least one additional operation. Additional operations may include operation 1202, operation 1204, operation 1206, and/or operation 1208.

Operation 1202 illustrates an indication of an unregulated inhalation. For example, as shown in FIGS. 1 through 5, algorithm utilizer module 440 may utilize an algorithm for recommending at least one artificial sensory experience. An algorithm for recommending an artificial sensory experience may include any computation, formula, statistical survey, and/or look-up table for determining and/or selecting a suitable artificial sensory experience. Some examples may include a computer software algorithm, a calculator, a flowchart, and/or a decision tree. In one embodiment, algorithm utilizer module 440 may utilize an algorithm that uses an inputted indication of an analgesic, such as oxycodone, and determines a suitable artificial sensory experience by analyzing periods of low blood concentration of the oxycodone. In this embodiment, algorithm utilizer module 440 may recommend an artificial sensory experience that may be effective in pain distraction when bioactive agent blood concentration may be reduced but before an additional dose may be available. In some instances, algorithm utilizer module 440 may include a computer processor.

Further, operation 1204 illustrates an indication of an unregulated inhalation. For example, as shown in FIGS. 1 through 5, contraindication algorithm utilizer module 442 may utilize an algorithm configured for identifying a contraindication of the artificial sensory experience. A contraindication of an artificial sensory experience may include giving an indication against the advisability of the artificial sensory experience. For example, contraindication algorithm utilizer module 442 may utilize an algorithm that considers an individual's personal medical history, such as a phobia, and may recommend not prescribing a certain artificial sensory experience, which may include an object that may trigger the phobia. Contraindication algorithm utilizer module 442 may identify a contraindication of an artificial sensory experience for reasons such as an adverse effect and/or inefficacy. In some instances, contraindication algorithm utilizer module 442 may include a computer processor.

Operation 1206 illustrates presenting an indication of an artificial sensory experience at least partly based on a personal medical history. For example, as shown in FIGS. 1 through 5, medical history indication presenter module 444 may present an indication of an artificial sensory experience at least partly based on a personal medical history. A medical history may include a personal history and/or a family history. A personal medical history may include a list of previous illnesses, symptoms, medicines, treatments, health risk factors, operations, and/or doctor visits associated with at least one individual. A personal and/or a family medical history may include life history and/or social history characteristics such as smoking, drinking, drug use, sexual history, exercise history, eating history, nutraceutical history, or the like. In one embodiment, medical history indication presenter module 444 may present an indication of a suitable virtual world based on a personal medical history. In this embodiment, the personal medical history may indicate that an individual may be averse to a certain virtual world, such as a virtual world with rapid animation that may cause nausea. In some instances, medical history indication presenter module 444 may include a computer processor and/or a display device, such as a computer monitor and/or a printer.

Operation 1208 illustrates utilizing an algorithm configured for recommending at least one of an artificial sensory experience. For example, as shown in FIGS. 1 through 5, experimental data indication presenter module 446 may present an indication of an artificial sensory experience at least partly based on experimental data. Experimental data may include any data from an experiment, such as a clinical trial. The experiment may be an experiment including an individual and/or a group of people. In one embodiment, experimental data indication presenter module 446 may present an indication of a virtual world suitable for an individual based on a clinical trial involving a group of 1,000 people showing a certain success rate for reducing a phobia, such as fear of heights. In some instances, experimental data indication presenter module 446 may include a computer processor and/or a display device, such as a computer monitor, a mobile phone, and/or a printer.

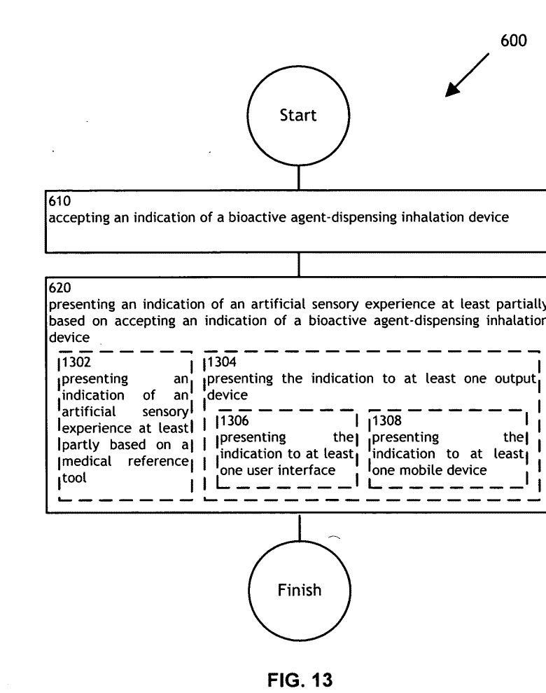

FIG. 13 illustrates alternative embodiments of the example operational flow 600 of FIG. 6. FIG. 13 illustrates example embodiments where the operation 620 may include at least one additional operation. Additional operations may include an operation 1302, an operation 1304, an operation 1306, and/or an operation 1308.

Operation 1302 illustrates presenting at least one of an indication of an artificial sensory experience or an indication of inhalation therapy at least partly based on a medical reference tool. For example, as shown in FIGS. 1 through 5, reference tool indication presenter module 448 may present an indication of an artificial sensory experience at least partly based on a medical reference tool. A medical reference tool may include a reference book, a reference database, and/or reference software. Some examples of a medical reference book may include a medical dictionary, a medical journal, and/or a book of drug interactions. One example of a reference database may include the National Cancer Center Cancer Image Reference (NCC-CIR) database and/or DynaMed. Some examples of reference software may include Skyscape software for a mobile phone and/or MedAlert. In one embodiment, reference tool indication presenter module 448 may present an indication of an artificial sensory experience based on a reference database, such as a database including data from a clinical trial. In some instances, reference tool indication presenter module 448 may include a computer processor and/or a display device, such as a mobile phone, a printer, and/or a computer monitor.

Operation 1304 illustrates presenting the indication to at least one output device. For example, as shown in FIGS. 1 through 5, output device presenter module 450 may present to at least one output device. In one example, output device presenter module 450 may present an indication of a combination prescription medication and an artificial sensory experience therapy to an output device 130, such as a printer and/or monitor at a health clinic. An output device may include any hardware device configured for receiving computer output. Some examples of an output device may include a printer, a monitor, a mobile phone, a speaker, and/or a visual display unit. The output device 130 may be used by individual 134. In some instances, output device presenter module 450 may include a computer processor.

Further, operation 1306 illustrates presenting the indication to at least one user interface. For example, as shown in FIGS. 1 through 5, user interface presenter module 452 may present to at least one user interface. In one embodiment, user interface presenter module 452 may present to a touchscreen device. A user interface may include means by which an individual may interact with a system. Some examples of a user interface may include a touchscreen, a graphical user interface, a tactile interface, and/or a live user interface. In some instances, user interface presenter module 452 may include a computer processor.

Further, operation 1308 illustrates presenting the indication to at least one mobile device. For example, as shown in FIGS. 1 through 5, mobile device presenter module 454 may present to at least one mobile device. In one embodiment, mobile device presenter module 454 may present to a mobile phone. A mobile device may include a portable computing device and may have wireless connection capability. Some examples of a mobile device may include a laptop or notebook computer, a personal digital assistant (PDA), an ipod, a smartphone, an Enterprise digital assistant (EDA), and/or a pager. In some instances, mobile device presenter module 454 may include a computer processor.

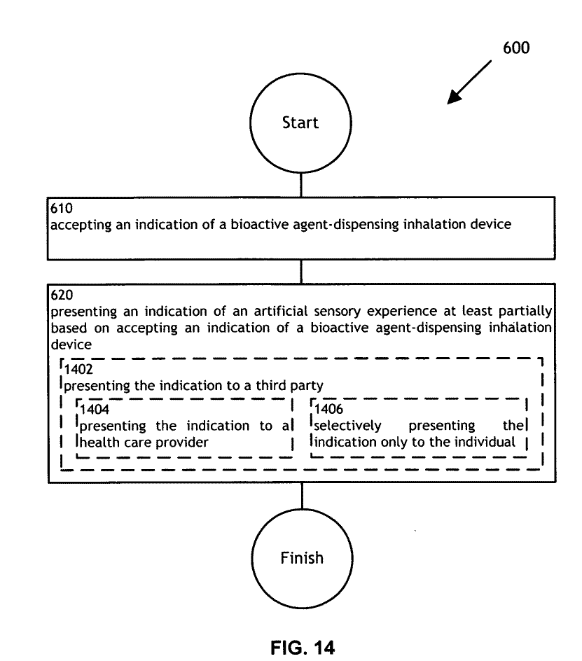

FIG. 14 illustrates alternative embodiments of the example operational flow 600 of FIG. 6. FIG. 14 illustrates example embodiments where operation 620 may include at least one additional operation. Additional operations may include operation 1402, operation 1404, and/or operation 1406.

Operation 1402 illustrates presenting the indication to at least one third party. For example, as shown in FIGS. 1 through 5, third party presenter module 456 may present to an individual's physician. A third party may include a party that is an independent party, person, and/or entity. Some examples of a third party may include a physician, a medical database, a hospital, a law enforcement agency, and/or a pharmacy. In one embodiment, third party presenter module 456 may present an indication to an insurance company. Another example of reporting to a third party may include creating displays and reports for aggregating data from therapy results, further discussed in Bair et al., U.S. Pat. No. 6,067,523, which is incorporated herein by reference. In some instances, third party presenter module 456 may include a computer processor and/or a communications device, such as a monitor and network link.

Further, operation 1404 illustrates presenting the indication to at least one health care provider. For example, as shown in FIGS. 1 through 5, health care provider presenter module 458 may present to a health care provider. A health care provider may include a pharmacy, a pharmaceutical company, a medical device company, a research institution, a computer software and/or computer hardware company, a website, a nurse and/or a physician. In one embodiment, health care provider presenter module 458 may present to a physician a prescribed combination artificial sensory experience and bioactive agent therapy via a secured website. In some instances, health care provider presenter module 458 may include a computer processor.

Further, operation 1406 illustrates selectively presenting the indication only to the individual. For example, as shown in FIGS. 1 through 5, selective presenter module 460 may selectively present only to the individual. Selective presenting may include limiting and/or blocking access of an individual's compliance results and/or a prescribed therapy, such as a prescribed artificial sensory experience and/or bioactive agent to a specific party. For example, selective presenter module 460 may present only to individual 134 and may keep results of a certain combination therapy confidential. In one embodiment, an encryption key may be employed to protect selected information. In an additional example, selective presenter module 460 may report only to a law enforcement agency and/or representative, such as a probation officer, and not to individual 134. In some instances, selective presenter module 460 may include a computer processor.

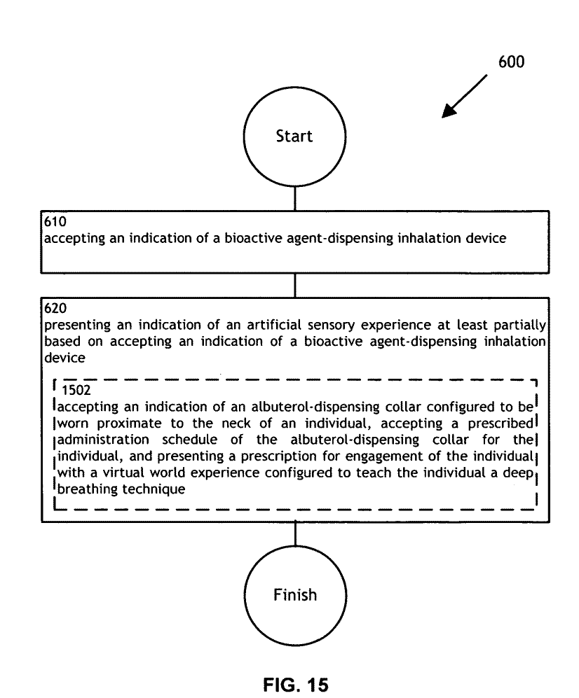

FIG. 15 illustrates alternative embodiments of the example operational flow 600 of FIG. 6. FIG. 15 illustrates example embodiments where the operation 620 may include at least one additional operation. Additional operations may include an operation 1502.

Operation 1502 illustrates accepting an indication of an individual's asthma, presenting a prescribed administration schedule of an albuterol-dispensing collar therapy for the individual, and presenting a prescription for engagement of the individual with a virtual world experience configured to teach the individual a deep breathing technique. For example, as shown in FIGS. 1 through 5, accepter module 102 and/or presenter module 104 may accept an indication of an albuterol-dispensing collar configured to be worn proximate to the neck of an individual, accept a prescribed administration schedule of the albuterol-dispensing collar for the individual, and present a prescription for engagement of the individual with a virtual world experience configured to teach the individual a deep breathing technique. In some instances, accepter module 102 and/or presenter module 104 may include a computer processor.

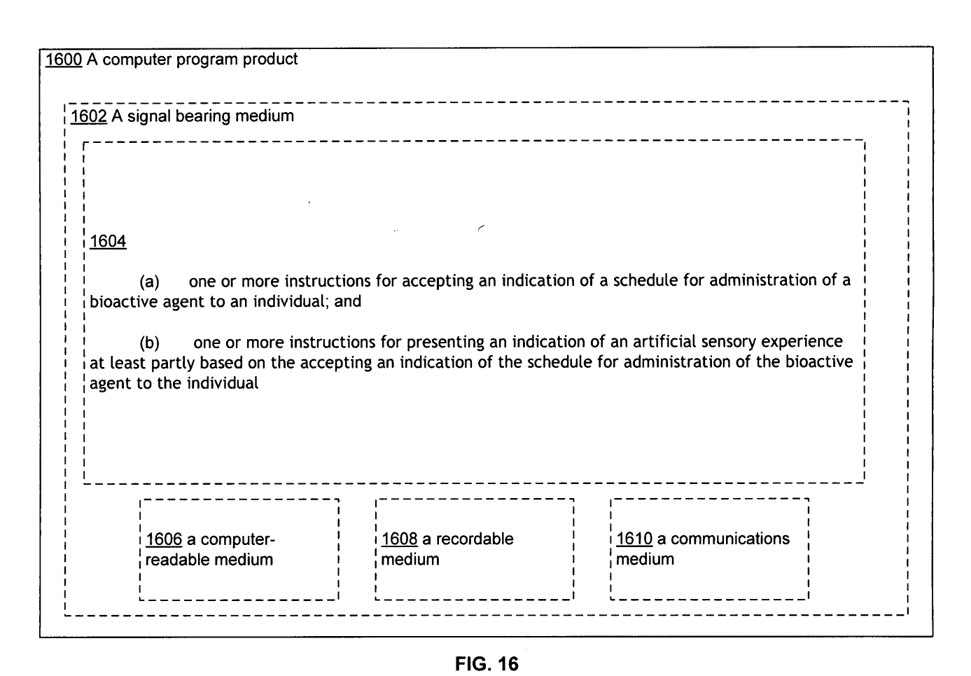

FIG. 16 illustrates a partial view of an example computer program product 1600 that includes a computer program 1604 for executing a computer process on a computing device. An embodiment of the example computer program product 1600 is provided using a signal-bearing medium bearing 1602, and may include one or more instructions for accepting an indication of at least one health-related condition and one or more instructions for presenting an indication of at least one artificial sensory experience and an indication of at least one inhalation therapy at least partially based on the accepting at least one indication of a health-related condition. The one or more instructions may be, for example, computer executable and/or logic-implemented instructions. In one implementation, the signal-bearing medium 1602 may include a computer-readable medium 1606. In one implementation, the signal bearing medium 1602 may include a recordable medium 1608. In one implementation, the signal bearing medium 1602 may include a communications medium 1610.

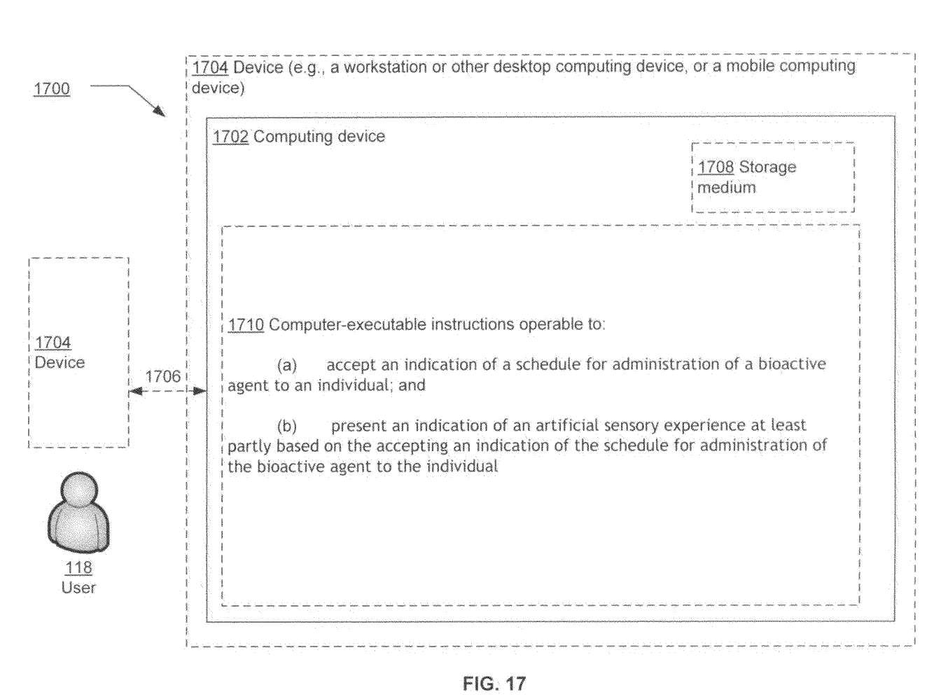

FIG. 17 illustrates an example system 1700 in which embodiments may be implemented. The system 1700 includes a computing system environment. The system 1700 also illustrates the user 118 using a device 1704, which is optionally shown as being in communication with a computing device 1702 by way of an optional coupling 1706. The optional coupling 1706 may represent a local, wide-area, or peer-to-peer network, or may represent a bus that is internal to a computing device (e.g., in example embodiments in which the computing device 1702 is contained in whole or in part within the device 1704). A storage medium 1708 may be any computer storage media.

The computing device 1702 includes computer-executable instructions 1710 that when executed on the computing device 1702 cause the computing device 1702 to accept an indication of a schedule for administration of a bioactive agent to an individual and present an indication of an artificial sensory experience at least partly based on the accepting an indication of the schedule for administration of the bioactive agent to the individual. As referenced above and as shown in FIG. 17, in some examples, the computing device 1702 may optionally be contained in whole or in part within the device 1704.

In FIG. 17, then, the system 1700 includes at least one computing device (e.g., 1702 and/or 1704). The computer-executable instructions 1710 may be executed on one or more of the at least one computing device. For example, the computing device 1702 may implement the computer-executable instructions 1710 and output a result to (and/or receive data from) the computing device 1704. Since the computing device 1702 may be wholly or partially contained within the computing device 1704, the device 1704 also may be said to execute some or all of the computer-executable instructions 1710, in order to be caused to perform or implement, for example, various ones of the techniques described herein, or other techniques.

The device 1704 may include, for example, a portable computing device, workstation, or desktop computing device. In another example embodiment, the computing device 1702 is operable to communicate with the device 1704 associated with the user 118 to receive information about the input from the user 118 for performing data access and data processing and presenting an output of the user-health test function at least partly based on the user data.

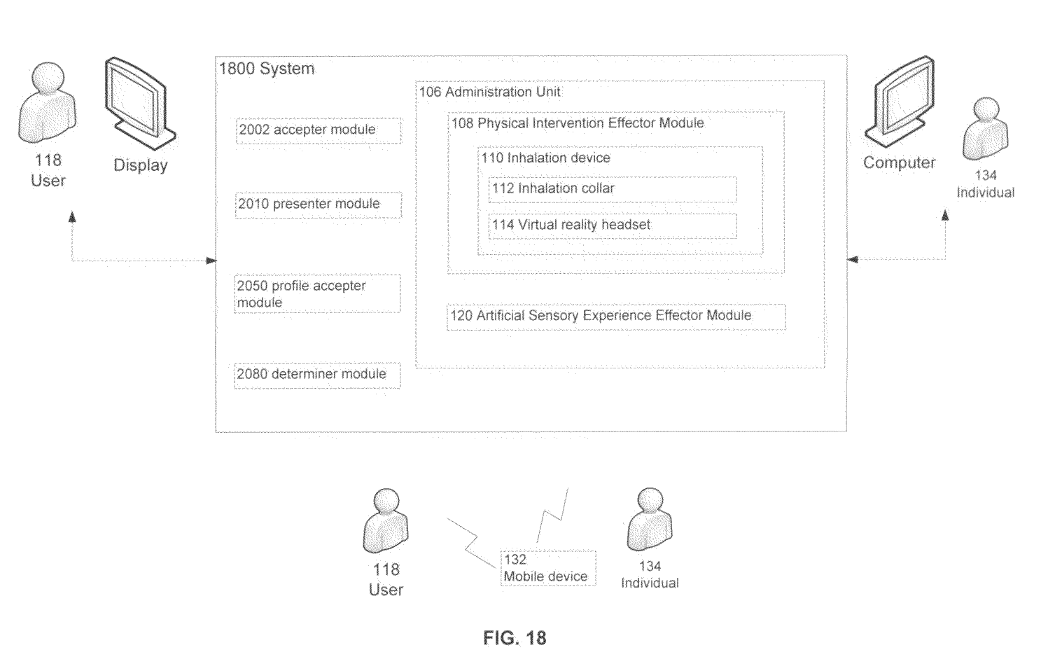

FIG. 18 illustrates system 1800 for accepting an indication of a schedule for administration of an inhalation device-dispensed bioactive agent to an individual and/or presenting an indication of an artificial sensory experience at least partly based on the accepting an indication of the schedule for administration of the inhalation device-dispensed bioactive agent to the individual. System 1800 may include accepter module 2002, presenter module 2010, profile accepter module 2050, determiner module 2080, and/or administration unit 106. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, system 1800 may include mobile device 132.

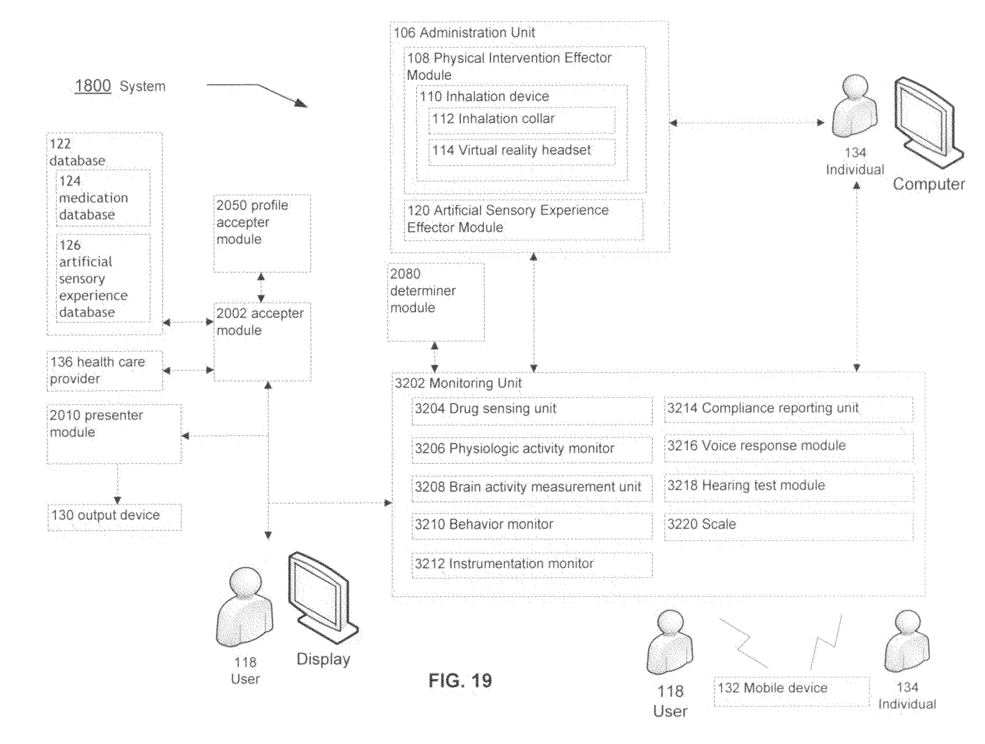

FIG. 19 illustrates system 1800 for accepting an indication of a schedule for administration of an inhalation device-dispensed bioactive agent to an individual and/or presenting an indication of an artificial sensory experience at least partly based on the accepting an indication of the schedule for administration of the inhalation device-dispensed bioactive agent to the individual. System 1800 may include accepter module 2002, presenter module 2010, profile accepter module 2050, determiner module 2080, accepter module 2002, presenter module 2010, profile accepter module 2050, and/or determiner module 2080 administration unit 106, and/or monitoring unit 3202. Accepter module 2002 may receive and/or transmit information and/or data to and/or from user 118, database 122, output device 130, and/or health care provider 136. A user may include user 118, individual 134, health care provider 136, a patient, and/or another affected person or entity. Database 122 may include medication database 124 and/or artificial sensory experience database 126. Monitoring unit 3202 may monitor individual 134 and may include drug sensing unit 3204, physiologic activity monitor 3206, brain activity measurement unit 3208, behavior monitor 3210, instrumentation monitor 3212, compliance reporting unit 3214, voice response module 3216, hearing test module 3218, and/or scale 3220. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, mobile device 132 may communicate with accepter module 2002, presenter module 2032, healthcare provider 136, user 118, individual 134, monitoring unit 3202, and/or administration unit 106.

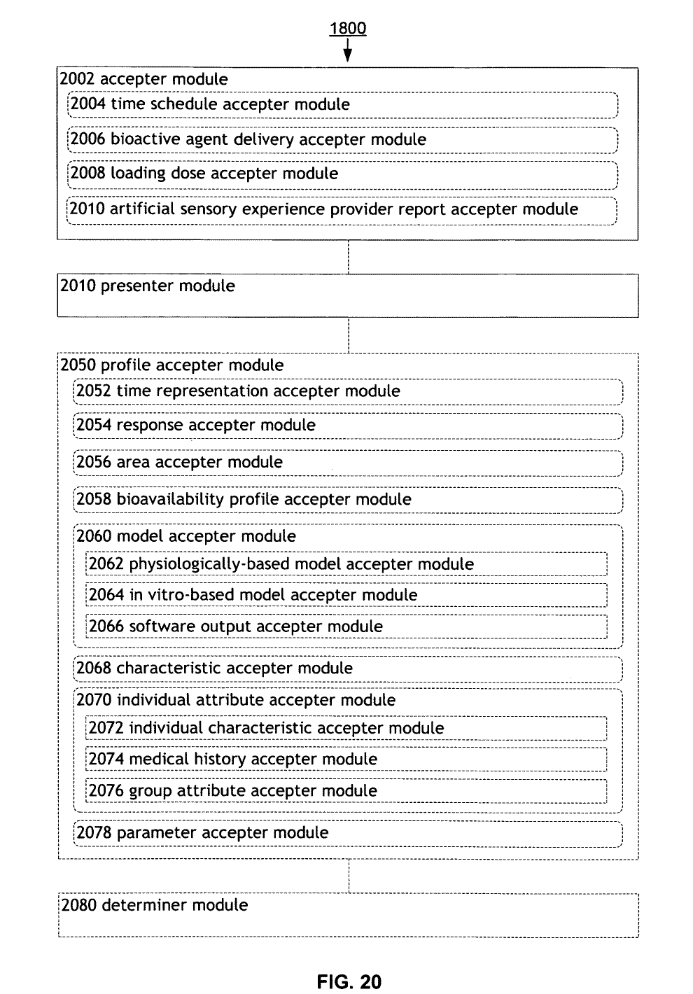

FIG. 20 further illustrates system 1800 including accepter module 2002, presenter module 2010, profile accepter module 2050, and/or determiner module 2080. Accepter module 2002 may include time schedule accepter module 2004, bioactive agent delivery accepter module 2006, loading dose accepter module 2008, and/or artificial sensory experience provider report accepter module 2010. Profile accepter module 2050 may include time representation accepter module 2052, response accepter module 2054, area accepter module 2056, bioavailability profile accepter module 2058, model accepter module 2060, characteristic accepter module 2068, individual attribute accepter module 2070, and/or parameter accepter module 2078. Model accepter module 2060 may include physiologically-based model accepter module 2062, in vitro-based model accepter module 2064, and/or software output accepter module 2066. Individual attribute accepter module 2070 may include individual characteristic accepter module 2072, medical history accepter module 2074, and/or group attribute accepter module 2076.

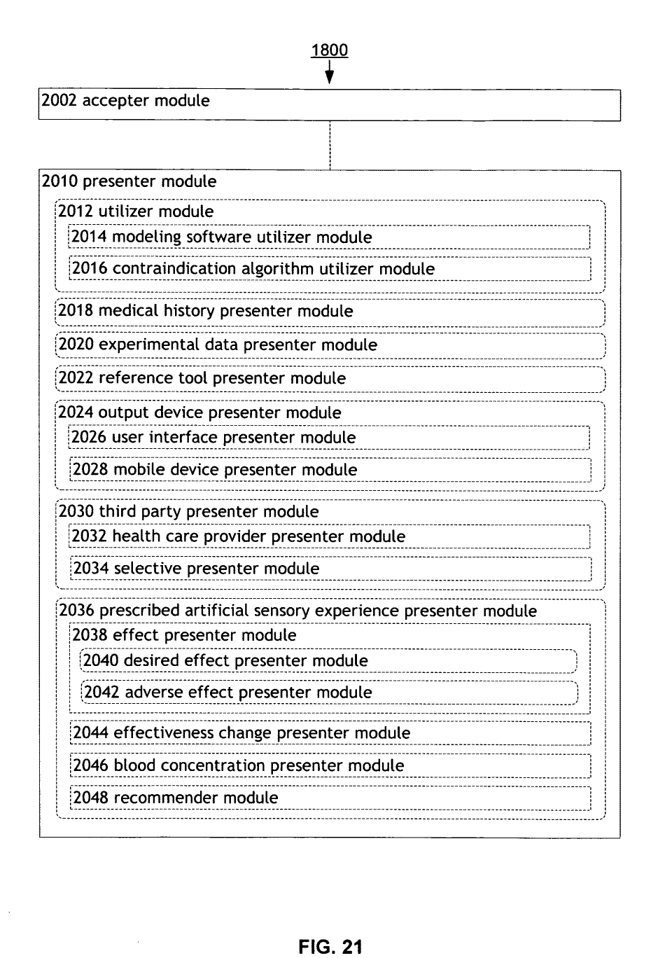

FIG. 21 further illustrates system 1800 including accepter module 2002, presenter module 2010, profile accepter module 2050, and/or determiner module 2080. Presenter module 2010 may include utilizer module 2012, medical history presenter module 2018, experimental data presenter module 2020, reference tool presenter module 2022, output device presenter module 2024, third party presenter module 2030, and/or prescribed artificial sensory experience presenter module 2036. Utilizer module 2012 may include modeling software utilizer module 2014 and/or contraindication algorithm utilizer module 2016. Output device presenter module 2024 may include user interface presenter module 2026 and/or mobile device presenter module 2028. Third party presenter module 2030 may include health care provider presenter module 2032 and/or selective presenter module 2034. Prescribed artificial sensory experience presenter module 2036 may include effect presenter module 2038, effectiveness change presenter module 2044, blood concentration presenter module 2046, and/or recommender module 2048. Effect presenter module 2038 may include desired effect presenter module 2040 and/or adverse effect presenter module 2042.

System 1800 generally represents instrumentality for accepting an indication of a schedule for administration of an inhalation device-dispensed bioactive agent to an individual and presenting an indication of an artificial sensory experience at least partly based on the accepting an indication of the schedule for administration of the inhalation device-dispensed bioactive agent to the individual. The operations of accepting an indication of a schedule for administration of an inhalation device-dispensed bioactive agent to an individual and presenting an indication of an artificial sensory experience at least partly based on the accepting an indication of the schedule for administration of the inhalation device-dispensed bioactive agent to the individual may be accomplished electronically, such as with a set of interconnected electrical components, an integrated circuit, and/or a computer processor.

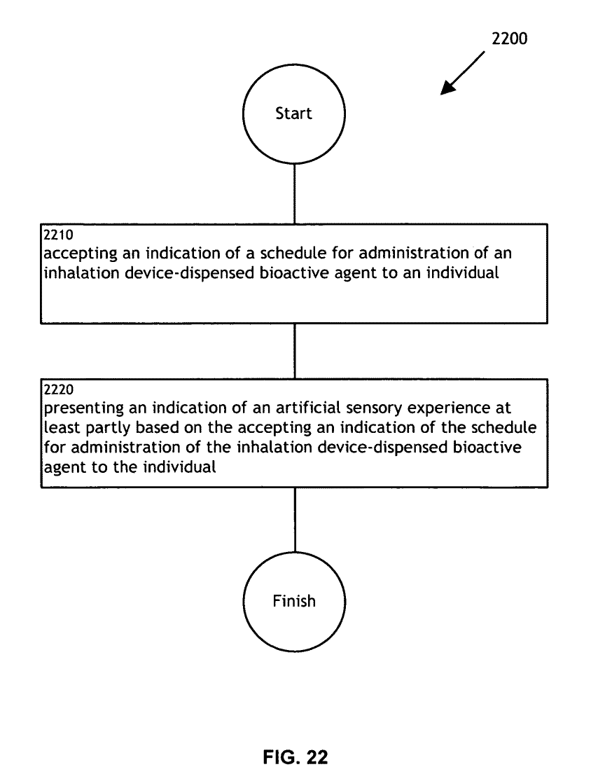

FIG. 22 illustrates an operational flow 2200 representing example operations related to accepting an indication of a schedule for administration of an inhalation device-dispensed bioactive agent to an individual and presenting an indication of an artificial sensory experience at least partly based on the accepting an indication of the schedule for administration of the inhalation device-dispensed bioactive agent to the individual. In FIG. 22 and in following figures that include various examples of operational flows, discussion and explanation may be provided with respect to the above-described examples of FIGS. 18 through 21, and/or with respect to other examples and contexts. However, it should be understood that the operational flows may be executed in a number of other environments and contexts, and/or in modified versions of FIGS. 18 through 21. Also, although the various operational flows are presented in the sequence(s) illustrated, it should be understood that the various operations may be performed in other orders than those which are illustrated, or may be performed concurrently.

After a start operation, the operational flow 2200 moves to an operation 2210. Operation 2210 depicts accepting an indication of a schedule for administration of an inhalation device-dispensed bioactive agent to an individual. For example, as shown in FIGS. 18 through 21, accepter module 2002 may accept an indication of a schedule for administration of an inhalation device-dispensed bioactive agent to an individual. In one embodiment, accepter module 2002 may accept an indication of a schedule for an inhalational administration of a bronchodilator to an individual. In this embodiment, the schedule may include specific times and/or methods that the inhalation device-dispensed bioactive agent may be administered. For example, a time schedule may specify that an individual should receive a specific dose of albuterol every two hours. In another example, an administration schedule may specify that an asthma medication should be administered intravenously at night and inhaled during waking hours. In some instances, accepter module 2002 may include a computer processor and/or a user interface coupled to the computer processor.

Then, operation 2220 depicts presenting an indication of an artificial sensory experience at least partly based on the accepting an indication of the schedule for administration of the inhalation device-dispensed bioactive agent to the individual. For example, as shown in FIGS. 18 through 21, presenter module 2010 may present an indication of an artificial sensory experience at least partly based on the accepting an indication of the schedule for administration of the inhalation device-dispensed bioactive agent to the individual. In one embodiment, presenter module 2010 may present an indication of a virtual world configured for distracting an individual at least partly based on accepting an indication of an inhalation device-dispensed bioactive agent administration time schedule. In this embodiment, the inhalation device-dispensed bioactive administration schedule may be coordinated so that an artificial sensory experience is administered when an inhalation device-dispensed bioactive agent may be less effective. Coordinating an artificial sensory experience administration schedule may serve, for example, to more efficiently distract an individual during a period of low bioactive agent concentration in an individual's blood so that the individual's attention may not be focused on a disorder and/or physical disability. In some instances, presenter module 2010 may include a computer processor, a display, and/or a printer.

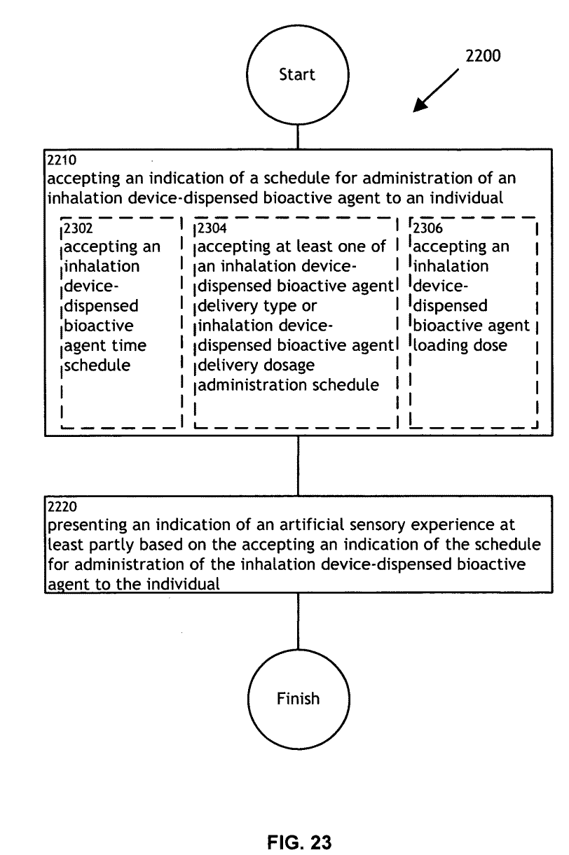

FIG. 23 illustrates alternative embodiments of the example operational flow 2200 of FIG. 22. FIG. 23 illustrates example embodiments where operation 2210 may include at least one additional operation. Additional operations may include operation 2302, operation 2304, and/or operation 2306.

Operation 2302 illustrates accepting an inhalation device-dispensed bioactive agent time schedule. For example, as shown in FIGS. 18 through 21, time schedule accepter module 2004 may accept an inhalation device-dispensed bioactive agent time schedule. In one embodiment, time schedule accepter module 2004 may accept an inhalation device-dispensed bioactive agent time schedule which specifies that an asthma medication should be administered every two hours. An inhalation device-dispensed bioactive agent time schedule may specify, for example, the exact and/or appropriate time an inhalation device-dispensed bioactive agent should be administered. Additionally, an inhalation device-dispensed bioactive agent time schedule may specify different inhaled bioactive agents and/or combinations of bioactive agents, delivery methods for administering an inhalation device-dispensed bioactive agent, and/or an inhalation device-dispensed bioactive agent dosage. Accepting an inhalation device-dispensed bioactive agent time schedule may include accepting from a computer processor, accepting from a memory device, and/or accepting from a user input. In one example, time schedule accepter module 2004 may accept an inhalation device-dispensed bioactive agent time schedule configured for printing on a medication blister pack, such as that described in Steinnagel, U.S. Pat. No. 4,974,729, which is incorporated herein by reference. In an additional example, time schedule accepter module 2004 may accept an inhalation device-dispensed bioactive agent time schedule from a device configured to remind an individual when to take medication, such as that in Goetz, U.S. Pat. No. 6,314,384, which is incorporated herein by reference. In some instances, time schedule accepter module 2004 may include a computer processor.

Operation 2304 illustrates accepting at least one of an inhalation device-dispensed bioactive agent delivery type or inhalation device-dispensed bioactive agent delivery dosage administration schedule. For example, as shown in FIGS. 18 through 21, bioactive agent delivery accepter module 2006 may accept at least one of an inhalation device-dispensed bioactive agent delivery type schedule or an inhalation device-dispensed bioactive agent delivery dosage administration schedule. Accepting an inhalation device-dispensed bioactive agent time schedule may include accepting from a computer processor, accepting from a memory device, and/or accepting from a user input. In one embodiment, bioactive agent delivery accepter module 2006 may accept an inhalation device-dispensed bioactive agent delivery type schedule specifying an administration of an inhaled asthmatic agent. In this embodiment, the asthmatic bioactive agent time schedule may indicate that the asthmatic agent only is administered inhalationally. In another embodiment, bioactive agent delivery accepter module 2006 may accept an inhalation device-dispensed bioactive agent delivery dosage specifying a bronchodilator administration dosage for a first time period, such as during an individual's awake hours, and a second bronchodilator administration dosage for a second time period, such as during an individual's sleeping hours. In some instances, bioactive agent delivery accepter module 2006 may include a computer processor.

Operation 2306 illustrates accepting an inhalation device-dispensed bioactive agent loading dose. For example, as shown in FIGS. 18 through 21, loading dose accepter module 2008 may accept an inhalation device-dispensed bioactive agent loading dose. A loading dose may include an initial dose that is higher than a maintenance and/or average dose. Often, a loading dose may be used for an inhalation device-dispensed bioactive agent that is slowly eliminated from an individual's body. In one embodiment, loading dose accepter module 2008 may accept a steroid loading dose for treatment of a bronchial infection. Some other bioactive agents a loading dose may be used with may include digoxin, teicoplanin, voriconazole, and/or procainamide. In some instances, loading dose accepter module 2008 may include a computer processor.

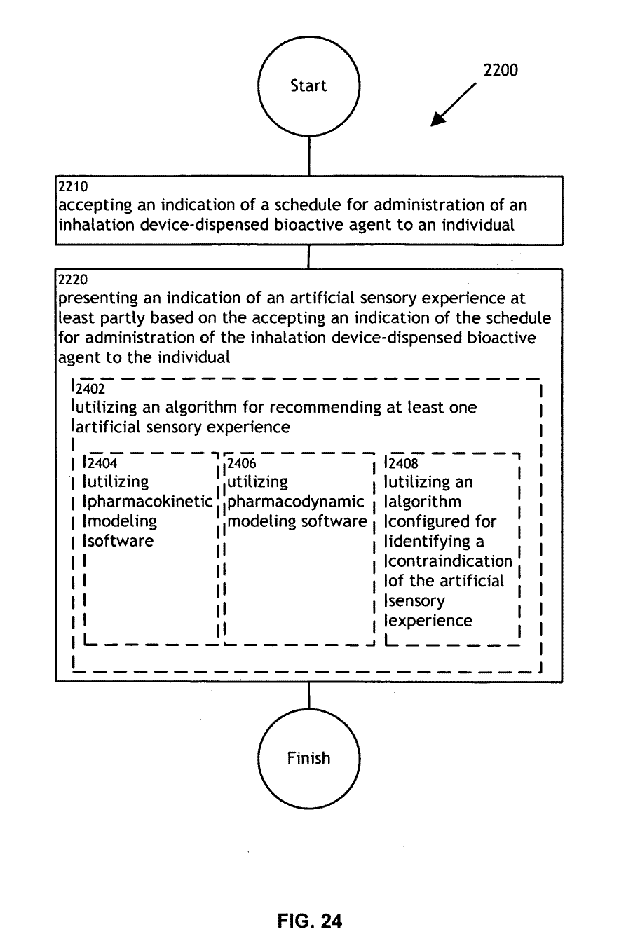

FIG. 24 illustrates alternative embodiments of the example operational flow 2200 of FIG. 22. FIG. 24 illustrates example embodiments where operation 2220 may include at least one additional operation. Additional operations may include operation 2402, operation 2404, operation 2406, and/or operation 2408.

Operation 2402 illustrates utilizing an algorithm for recommending at least one artificial sensory experience. For example, as shown in FIGS. 18 through 21, utilizer module 2012 may utilize an algorithm for recommending at least one artificial sensory experience. An algorithm for recommending an artificial sensory experience may include any computation, formula, statistical survey, and/or look-up table for determining and/or selecting a suitable artificial sensory experience. Some examples may include a computer software algorithm, a calculator, a flowchart, and/or a decision tree. In one embodiment, utilizer module 2012 may utilize an algorithm that uses an inputted indication of an analgesic, such as nitrous oxide, and determines a suitable artificial sensory experience by analyzing periods of low blood concentration of the nitrous oxide. In this embodiment, utilizer module 2012 may recommend an artificial sensory experience that may be effective in pain distraction when bioactive agent blood concentration may be reduced but before an additional dose may be available. In some instances, utilizer module 2012 may include a computer processor.

Further, operation 2404 illustrates utilizing pharmacokinetic modeling software. For example, as shown in FIGS. 18 through 21, modeling software utilizer module 2014 may utilize pharmacokinetic modeling software. In one embodiment, modeling software utilizer module 2014 may utilize modeling software to determine and display characteristics regarding a specific bioactive agent. Pharmacokinetic modeling software may include software configured for displaying pharmacokinetic information for a specific bioactive agent, such as a pharmacokinetic profile. Additionally, pharmacokinetic modeling software may analyze and compare a specific bioactive agent and/or a pharmacokinetic profile with an artificial sensory experience and make a recommendation based on, for example, low bioactive blood concentration. An additional example of utilizing pharmacokinetic modeling software may be found in Bachman et al., U.S. Patent Publication No. 2006/0161408, which is incorporated herein by reference. Some examples of pharmacokinetic software may include acslXtreme, available from Aegis Technologies Group, Inc., West Austin, Tex., and GastroPlus, available from Simulations Plus, Inc., Lancaster, Calif. In some instances, modeling software utilizer module 2014 may include a computer processor.

Further, operation 2406 illustrates utilizing pharmacodynamic modeling software. For example, as shown in FIGS. 18 through 21, modeling software utilizer module 2014 may utilize pharmacodynamic modeling software. In one embodiment, modeling software utilizer module 2014 may utilize pharmacodynamic modeling software for determining an appropriate artificial sensory experience based on how a specific bioactive agent, such as nitrous oxide, affects an individual. Utilizing pharmacodynamic software may be useful for determining an appropriate artificial sensory experience that may compensate for a period of low bioactive agent blood concentration. An appropriate artificial sensory experience may compensate for reduced bioactive agent efficacy by acting as a distraction when the bioactive agent concentration may be reduced. One example of pharmacodynamic modeling software may include GastroPlus, available from Simulations Plus, Inc., Lancaster, Calif. In some instances, modeling software utilizer module 2014 may include a computer processor.

Further, operation 2408 illustrates utilizing an algorithm configured for identifying a contraindication of the artificial sensory experience. For example, as shown in FIGS. 18 through 21, contraindication algorithm utilizer module 2016 may utilize an algorithm configured for identifying a contraindication of the artificial sensory experience. A contraindication of an artificial sensory experience may include giving an indication against recommending the artificial sensory experience. For example, contraindication algorithm utilizer module 2016 may utilize an algorithm that considers an individual's personal medical history, such as a phobia, and may recommend not prescribing a certain artificial sensory experience, which may include an object that may trigger the phobia. Contraindication algorithm utilizer module 2016 may identify a contraindication of an artificial sensory experience for reasons such as, for example, an adverse effect and/or inefficacy. In some instances, contraindication algorithm utilizer module 2016 may include a computer processor.

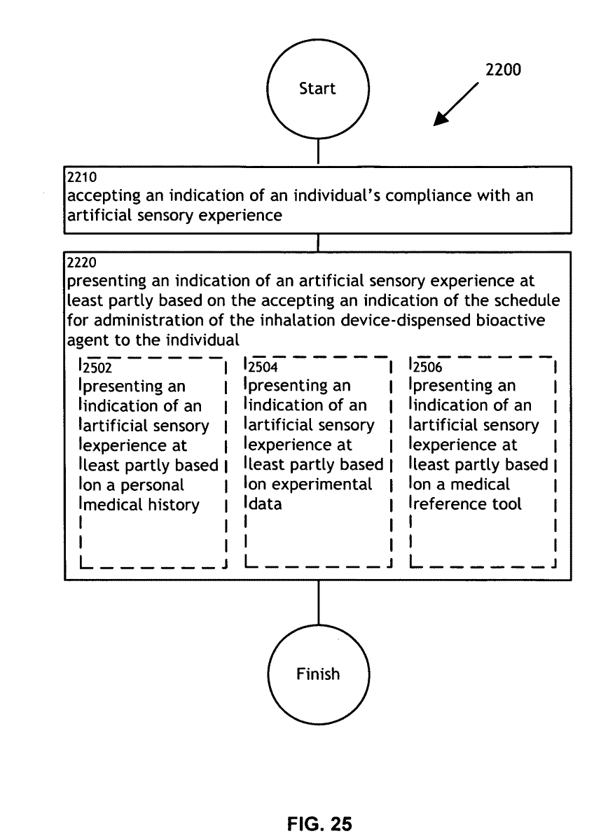

FIG. 25 illustrates alternative embodiments of the example operational flow 2200 of FIG. 22. FIG. 25 illustrates example embodiments where operation 2220 may include at least one additional operation. Additional operations may include operation 2502, operation 2504, and/or operation 2506.

Operation 2502 illustrates presenting an indication of an artificial sensory experience at least partly based on a personal medical history. For example, as shown in FIGS. 18 through 21, medical history presenter module 2018 may present an indication of an artificial sensory experience at least partly based on a personal medical history. A medical history may include a personal history and/or a family history. A personal medical history may include a list of previous illnesses, symptoms, medicines, treatments, health risk factors, operations, and/or doctor visits associated with at least one individual. A personal and/or a family medical history may include life history and/or social history characteristics such as smoking, drinking, drug use, sexual history, exercise history, eating history, nutraceutical history, or the like. In one embodiment, medical history presenter module 2018 may present an indication of a suitable virtual world based on a personal medical history. In this embodiment, the personal medical history may indicate that an individual may be averse to a certain virtual world, such as a virtual world with rapid animation that may cause nausea. In some instances, medical history presenter module 2018 may include a computer processor and/or a display device, such as a computer monitor and/or a printer.

Operation 2504 illustrates presenting an indication of an artificial sensory experience at least partly based on experimental data. For example, as shown in FIGS. 18 through 21, experimental data presenter module 2020 may present an indication of an artificial sensory experience at least partly based on experimental data. Experimental data may include any data from an experiment, such as a clinical trial. The experiment may be an experiment including an individual and/or a group of people. In one embodiment, experimental data presenter module 2020 may present an indication of a virtual world suitable for an individual based on a clinical trial involving a group of 1,000 people showing a certain success rate for reducing a phobia, such as fear of heights. In some instances, experimental data presenter module 2020 may include a computer processor and/or a display device, such as a computer monitor, a mobile phone, and/or a printer.

Operation 2506 illustrates presenting an indication of an artificial sensory experience at least partly based on a medical reference tool. For example, as shown in FIGS. 18 through 21, reference tool presenter module 2022 may present an indication of an artificial sensory experience at least partly based on a medical reference tool. A medical reference toot may include a reference book, a reference database, and/or reference software. Some examples of a medical reference book may include a medical dictionary, a medical journal, and/or a book of drug interactions. One example of a reference database may include the National Cancer Center Cancer Image Reference (NCC-CIR) database and/or DynaMed. Some examples of reference software may include Skyscape software for a mobile phone and/or MedAlert. In one embodiment, reference tool presenter module 2022 may present an indication of an artificial sensory experience based on a reference database, such as a database including data from a clinical trial. In some instances, reference tool presenter module 2022 may include a computer processor and/or a display device, such as a mobile phone, a printer, and/or a computer monitor.

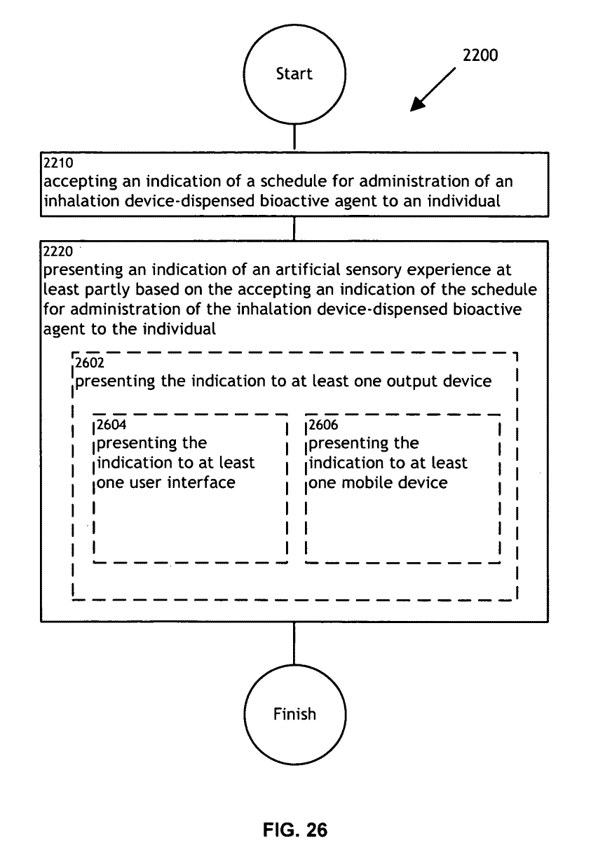

FIG. 26 illustrates alternative embodiments of the example operational flow 2200 of FIG. 22. FIG. 26 illustrates example embodiments where operation 2220 may include at least one additional operation. Additional operations may include operation 2602, operation 2604, and/or operation 2606.

Operation 2602 illustrates presenting the indication to at least one output device. For example, as shown in FIGS. 18 through 21, output device presenter module 2024 may present to at least one output device. In one example, output device presenter module 2024 may present an indication of a combination prescription medication and an artificial sensory experience therapy to an output device 130, such as a printer and/or monitor at a health clinic. An output device may include any hardware device configured for receiving computer output. Some examples of an output device may include a printer, a monitor, a mobile phone, a speaker, and/or a visual display unit. The output device 130 may be used by individual 134. In some instances, output device presenter module 2024 may include a computer processor.

Further, operation 2604 illustrates presenting the indication to at least one user interface. For example, as shown in FIGS. 18 through 21, user interface presenter module 2026 may present to at least one user interface. In one embodiment, user interface presenter module 2026 may present to a touchscreen device. A user interface may include means by which an individual may interact with a system. Some examples of a user interface may include a touchscreen, a graphical user interface, a tactile interface, and/or a live user interface. In some instances, user interface presenter module 2026 may include a computer processor.

Further, operation 2606 illustrates presenting the indication to at least one mobile device. For example, as shown in FIGS. 18 through 21, mobile device presenter module 2028 may present to at least one mobile device. In one embodiment, mobile device presenter module 2028 may present to a mobile phone. A mobile device may include a portable computing device and may have wireless connection capability. Some examples of a mobile device may include a laptop or notebook computer, a personal digital assistant (PDA), an ipod, a smartphone, an Enterprise digital assistant (EDA), and/or a pager. In some instances, mobile device presenter module 2028 may include a computer processor.

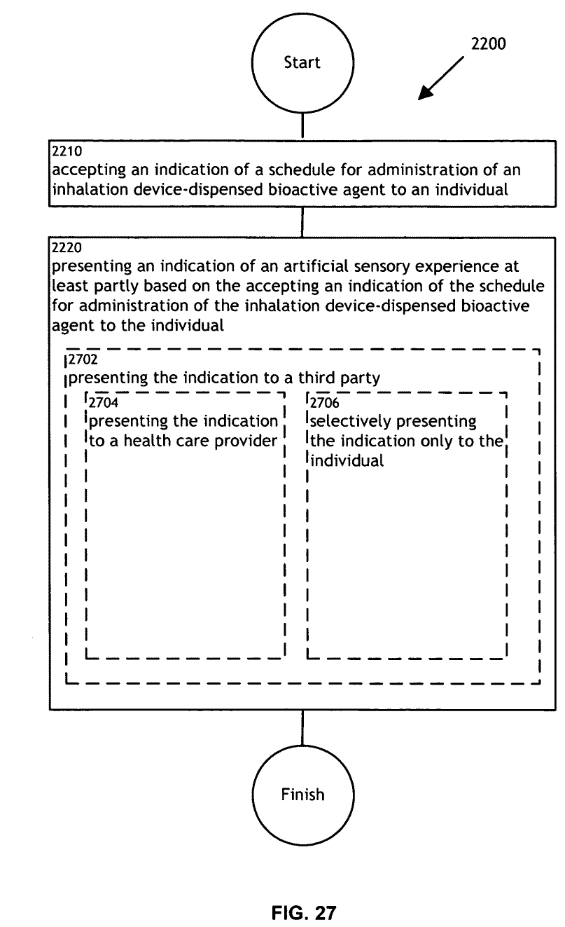

FIG. 27 illustrates alternative embodiments of the example operational flow 2200 of FIG. 22. FIG. 27 illustrates example embodiments where operation 2220 may include at least one additional operation. Additional operations may include operation 2702, operation 2704, and/or operation 2706.

Operation 2702 illustrates presenting the indication to a third party. For example, as shown in FIGS. 18 through 21, third party presenter module 2030 may present to an individual's physician. A third party may include a party that is an independent party, person, and/or entity. Some examples of a third party may include a physician, a medical database, a hospital, a law enforcement agency, and/or a pharmacy. One example of reporting to a third party may include creating displays and reports for aggregating data from therapy results, further discussed in Bair et al., U.S. Pat. No. 6,067,523, which is incorporated herein by reference. In some instances, third party presenter module 2030 may include a computer processor and/or a communications device, such as a monitor and network link.

Further, operation 2704 illustrates presenting the indication to a health care provider. For example, as shown in FIGS. 18 through 21, health care provider presenter module 2032 may present to a health care provider. A health care provider may include a pharmacy, a pharmaceutical company, a medical device company, a research institution, a computer software and/or computer hardware company, a website, a nurse and/or a physician. In one embodiment, health care provider presenter module 2032 may present to a physician a prescribed combination artificial sensory experience and bioactive agent therapy via a secured website. In some instances, health care provider presenter module 2032 may include a computer processor.

Further, operation 2706 illustrates selectively presenting the indication only to the individual. For example, as shown in FIGS. 18 through 21, selective presenter module 2034 may selectively present only to the individual. Selective presenting may include limiting and/or blocking access of an individual's compliance results and/or a prescribed therapy, such as a prescribed artificial sensory experience and/or inhalation device-dispensed bioactive agent to a specific party. For example, selective presenter module 2034 may present only to individual 134 and may keep results of a certain combination therapy confidential. In one embodiment, an encryption key may be employed to protect selected information. In an additional example, selective presenter module 3450 may report only to a law enforcement agency and/or representative, such as a probation officer, and not to individual 134. In some instances, selective presenter module 3450 may include a computer processor.

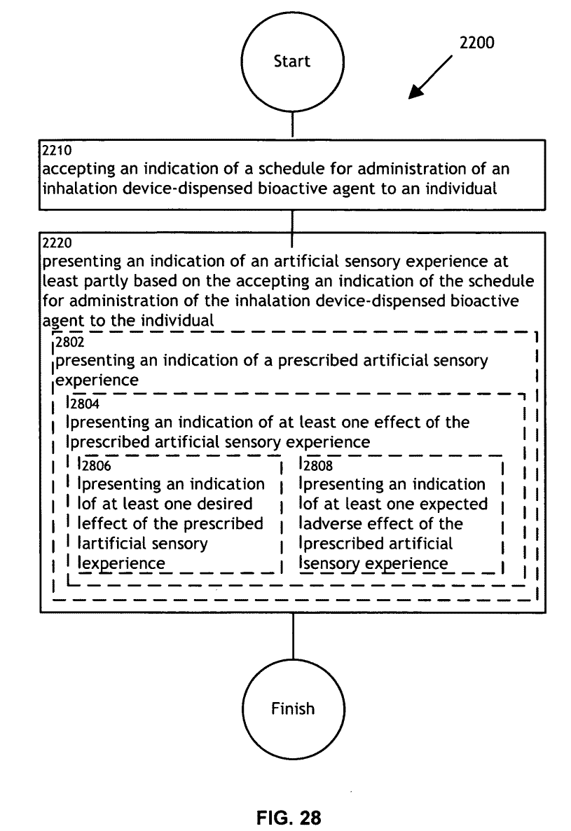

FIG. 28 illustrates alternative embodiments of the example operational flow 2200 of FIG. 22. FIG. 28 illustrates example embodiments where operation 2220 may include at least one additional operation. Additional operations may include operation 2802, operation 2804, operation 2806, and/or operation 2808.

Operation 2802 illustrates presenting an indication of a prescribed artificial sensory experience. For example, as shown in FIGS. 18 through 21, prescribed artificial sensory experience presenter module 2036 may present an indication of a prescribed artificial sensory experience. A prescribed artificial sensory experience may include any artificial sensory experience prescribed by a health care professional, such as a physician, a mental health specialist, a nurse, a physical therapist, an occupational therapist, a chiropractor, and/or a homeopathic practitioner. In one embodiment, prescribed artificial sensory experience presenter module 2036 may present an indication of a virtual world prescribed by a psychiatrist. In this embodiment, the prescribed virtual world may be configured to be administered in conjunction with a prescribed inhalation device-dispensed bioactive agent. Administering a prescribed inhalation device-dispensed bioactive agent in conjunction with a prescribed artificial sensory experience may serve to increase efficacy of the combined therapy, for example, by serving as a distraction from pain. In some instances, prescribed artificial sensory experience presenter module 2036 may include a computer processor and/or a display device, such as a computer monitor and/or a printer.

Further, operation 2804 illustrates presenting an indication of at least one effect of the prescribed artificial sensory experience. For example, as shown in FIGS. 18 through 21, effect presenter module 2038 may present an indication of at least one effect of the prescribed artificial sensory experience. In one embodiment, effect presenter module 2038 may present an indication of at least one effect of the prescribed artificial sensory experience. An effect may include a reaction and/or thing that occurs as a result of the artificial sensory experience. For example, an effect may include a side effect, a desired effect, and/or an adverse effect. Some examples of an effect may include an increased bioactive agent efficacy, dizziness, and/or a decreased heart rate. In some instances, effect presenter module 2038 may include a computer processor.

Further, operation 2806 illustrates presenting an indication of at least one desired effect of the prescribed artificial sensory experience. For example, as shown in FIGS. 18 through 21, desired effect presenter module 2040 may present an indication of at least one desired effect of the prescribed artificial sensory experience. Some examples of a desired effect may include effects such as an increased bioactive agent efficacy, a cured illness and/or condition, and/or a changed behavior. In one embodiment, desired effect presenter module 2040 may present an indication of an increased nitrous oxide efficacy measured by self pain evaluation by an individual. In some instances, desired effect presenter module 2040 may include a computer processor and/or a display, such as a monitor and/or a printer.

Further, operation 2808 illustrates presenting an indication of at least one expected adverse effect of the prescribed artificial sensory experience. For example, as shown in FIGS. 18 through 21, adverse effect presenter module 2042 may present an indication of an expected adverse effect of the prescribed artificial sensory experience. An adverse effect may include a harmful and/or undesired effect resulting from an intervention, such as an artificial sensory experience. Some examples of an adverse effect may include headache, dizziness, depression, bleeding, seizure, and/or fever. In one embodiment, adverse effect presenter module 2042 may present an indication of fever in an individual while being administered a prescribed artificial sensory experience and inhalation device-dispensed bioactive agent. In some instances, adverse effect presenter module 2042 may include a computer processor, a display device, such as a monitor and/or printer, and/or medical instrumentation, such as a thermometer configured for measuring a body temperature.

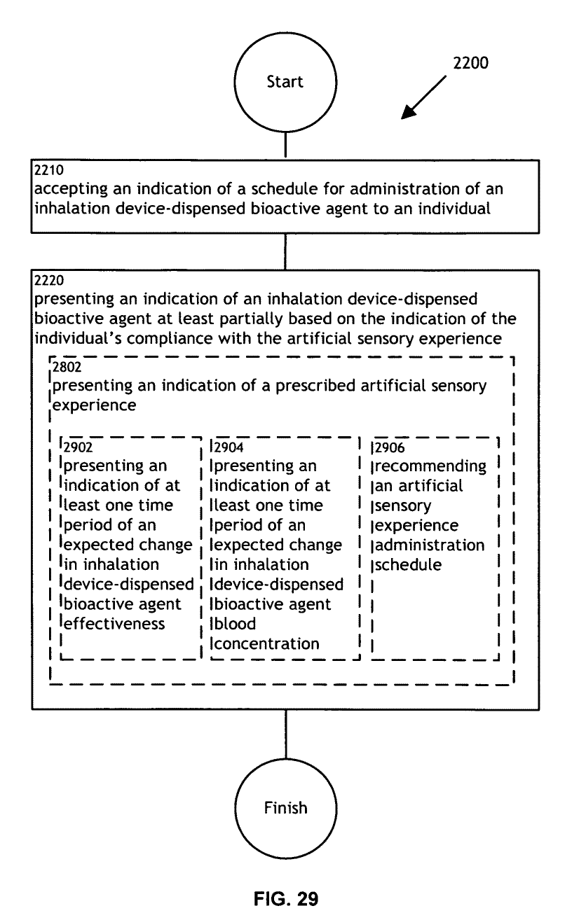

FIG. 29 illustrates alternative embodiments of the example operational flow 2200 of FIG. 22. FIG. 29 illustrates example embodiments where operation 2220 may include at least one additional operation. Additional operations may include operation 2902, operation 2904, and/or operation 2906.

Further, operation 2902 illustrates presenting an indication of at least one time period of an expected change in inhalation device-dispensed bioactive agent effectiveness. For example, as shown in FIGS. 18 through 21, effectiveness change presenter module 2044 may present an indication of at least one time period of an expected change in inhalation device-dispensed bioactive agent effectiveness. In one embodiment, effectiveness change presenter module 2044 may present an indication of a time period when nitrous oxide is expected to decrease in effectiveness. Such an indication of decrease and/or change in inhalation device-dispensed bioactive agent effectiveness may serve to indicate an appropriate time period for administering and/or modifying an artificial sensory experience to compensate for a change in inhalation device-dispensed bioactive agent efficacy. In another embodiment, effectiveness change presenter module 2044 may present an indication of a time period where a blood stream bronchodilator concentration drops. This time period of low blood stream bronchodilator concentration may be appropriate for presenting an immersive virtual world for serving as a distraction to any increase in coughing and/or breathing difficulty caused by a lowered bronchodilator concentration. In some instances, effectiveness change presenter module 2044 may include a computer processor.

Further, operation 2904 illustrates presenting an indication of at least one time period of an expected change in inhalation device-dispensed bioactive agent blood concentration. For example, as shown in FIGS. 18 through 21, blood concentration presenter module 2046 may present an indication of at least one time period of an expected change in inhalation device-dispensed bioactive agent blood concentration. In one embodiment, blood concentration presenter module 2046 may present an indication of a one hour time period of an expected change in nitrous oxide blood concentration. Indicating a time period of a change in blood concentration may serve to help determine an artificial sensory experience administration schedule. For example, if an inhalation device-dispensed bioactive agent blood concentration is expected to be reduced during a certain time period, an artificial sensory experience configured for distracting an individual from breathing difficulty may be selected for administration during that time period. In some instances, blood concentration presenter module 2046 may include a computer processor and/or a display device, such as a printer and/or a computer monitor.

Further, operation 2906 illustrates recommending an artificial sensory experience administration schedule. For example, as shown in FIGS. 18 through 21, recommender module 2048 may recommend an artificial sensory experience administration schedule. In one embodiment, recommender module 2048 may recommend a time schedule for administration of a virtual world experience. A time schedule may be recommended by taking into account factors involving the individual and/or the inhalation device-dispensed bioactive agent. For example, efficacy of the inhalation device-dispensed bioactive agent versus time may be a factor, such as a time period when the inhalation device-dispensed bioactive agent is less effective. Efficacy of the inhalation device-dispensed bioactive agent may be a factor in determining when an artificial sensory experience is administered because of the potential for the artificial sensory experience to compensate for a changed inhalation device-dispensed bioactive agent efficacy. An additional factor may include an attribute of the individual, such as how an inhalation device-dispensed bioactive agent and/or specific artificial sensory experience affects the individual, for example a side effect. Another example of recommending an artificial sensory experience may be found in Akazawa et al., U.S. Pat. No. 7,155,680, which is incorporated herein by reference. In some instances, recommender module 2048 may include a computer processor.

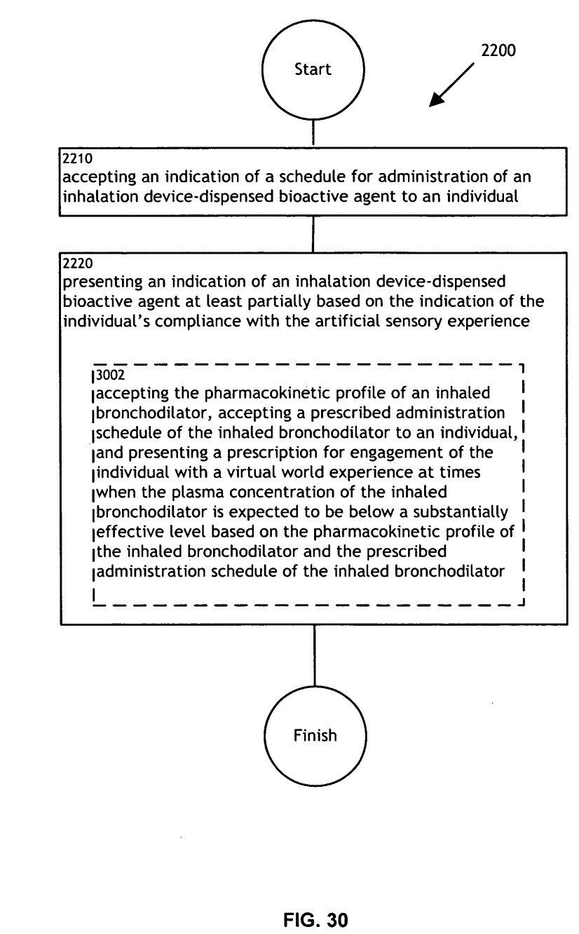

FIG. 30 illustrates alternative embodiments of the example operational flow 2200 of FIG. 22. FIG. 30 illustrates example embodiments where operation 2220 may include at least one additional operation. Additional operations may include an operation 3002.

Operation 3002 illustrates accepting the pharmacokinetic profile of an inhaled bronchodilator, accepting a prescribed administration schedule of the inhaled bronchodilator to an individual, and presenting a prescription for engagement of the individual with a virtual world experience at times when the plasma concentration of the inhaled bronchodilator is expected to be below a substantially effective level based on the pharmacokinetic profile of the inhaled bronchodilator and the prescribed administration schedule of the inhaled bronchodilator. For example, as shown in FIGS. 18 through 21, accepter module 2002 and/or presenter module 2010 may accept the pharmacokinetic profile of an inhaled bronchodilator, accepting a prescribed administration schedule of the inhaled bronchodilator to an individual, and presenting a prescription for engagement of the individual with a virtual world experience at times when the plasma concentration of the inhaled bronchodilator is expected to be below a substantially effective level based on the pharmacokinetic profile of the inhaled bronchodilator and the prescribed administration schedule of the inhaled bronchodilator. In some instances, accepter module 2002 may include a computer processor and/or a user interface coupled to the computer processor. In some instances, presenter module 2010 may include a computer processor, a display, and/or a printer.

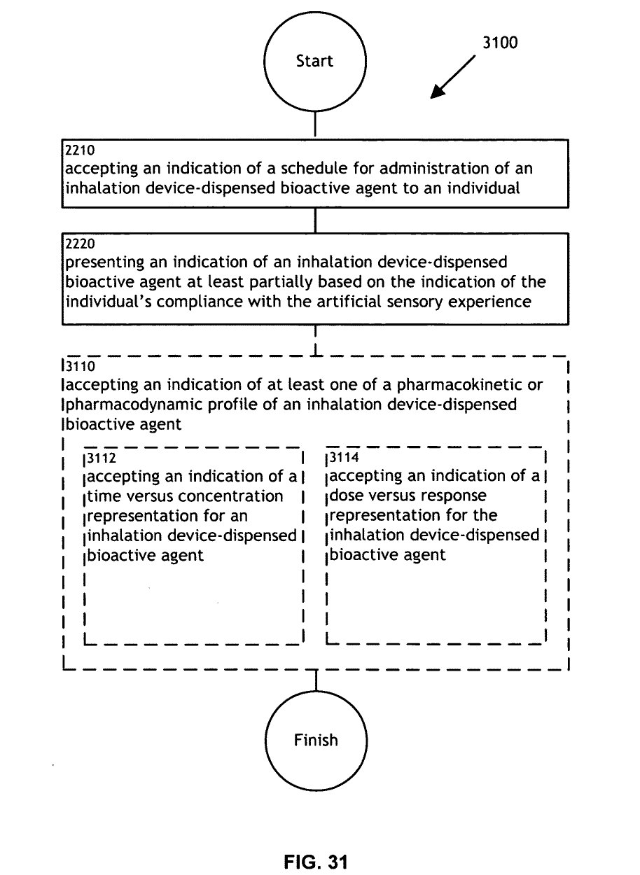

FIG. 31 illustrates an operational flow 3100 representing example operations related to accepting an indication of a schedule for administration of an inhalation device-dispensed bioactive agent to an individual, presenting an indication of an artificial sensory experience at least partly based on the accepting an indication of the schedule for administration of the inhalation device-dispensed bioactive agent to the individual, and accepting an indication of at least one of a pharmacokinetic or pharmacodynamic profile of an inhalation device-dispensed bioactive agent. FIG. 31 illustrates an example embodiment where the example operational flow 2200 of FIG. 22 may include at least one additional operation. Additional operations may include operation 3110, operation 3112, and/or operation 3114.

After a start operation, operation 2210, and operation 2220, the operational flow 3100 moves to operation 3110. Operation 3110 illustrates accepting an indication of at least one of a pharmacokinetic or pharmacodynamic profile of an inhalation device-dispensed bioactive agent. For example, as shown in FIGS. 18 through 21, profile accepter module 2050 may accept an indication of a pharmacokinetic and/or a pharmacodynamic profile of an inhalation device-dispensed bioactive agent. An inhalation device-dispensed bioactive agent pharmacokinetic profile may include information regarding how the inhalation device-dispensed bioactive agent is affected by the body. A pharmacokinetic profile may include information such as absorption, distribution, metabolism, and/or elimination. An inhalation device-dispensed bioactive agent pharmacodynamic profile may include information regarding how an inhalation device-dispensed bioactive agent is affected by the body, such as routes and/or mechanisms of inhalation device-dispensed bioactive agent absorption and excretion, the inhalation device-dispensed bioactive agent biotransformation in the body, and/or the rate of an inhalation device-dispensed bioactive agent action. In one embodiment, profile accepter module 2050 may accept an indication of a pharmacokinetic profile for nitrous oxide including, for example, a metabolism rate. In another embodiment, profile accepter module 2050 may accept an indication of a pharmacodynamic profile for halothane. In some instances, profile accepter module 2050 may include a computer processor and/or a user interface.

Operation 3112 illustrates accepting an indication of a time versus concentration representation for an inhalation device-dispensed bioactive agent. For example, as shown in FIGS. 18 through 21, time representation accepter module 2052 may accept an indication of a time versus concentration representation for an inhalation device-dispensed bioactive agent. In one embodiment, time representation accepter module 2052 may accept an indication of a time versus concentration representation for a regiment of prescribed halothane. A time versus concentration representation may serve to indicate time periods of reduced and/or changed inhalation device-dispensed bioactive agent effectiveness. A determination of reduced and/or changed inhalation device-dispensed bioactive agent effectiveness may indicate a suitable time period for administering an artificial sensory experience, which may serve as an additional distraction and/or compensation. In some instances, time representation accepter module 2052 may include a computer processor.

Operation 3114 illustrates accepting an indication of a dose versus response representation for the inhalation device-dispensed bioactive agent. For example, as shown in FIGS. 18 through 21, response accepter module 2054 may accept an indication of a dose versus response representation for an inhalation device-dispensed bioactive agent. In one embodiment, response accepter module 2054 may accept an indication of a dose versus response representation for sevoflurane. A dose versus response representation may serve to indicate a proper inhalation device-dispensed bioactive agent dosage for a desired response of an individual while the individual experiences an artificial sensory experience. In some instances, response accepter module 2054 may include a computer processor.

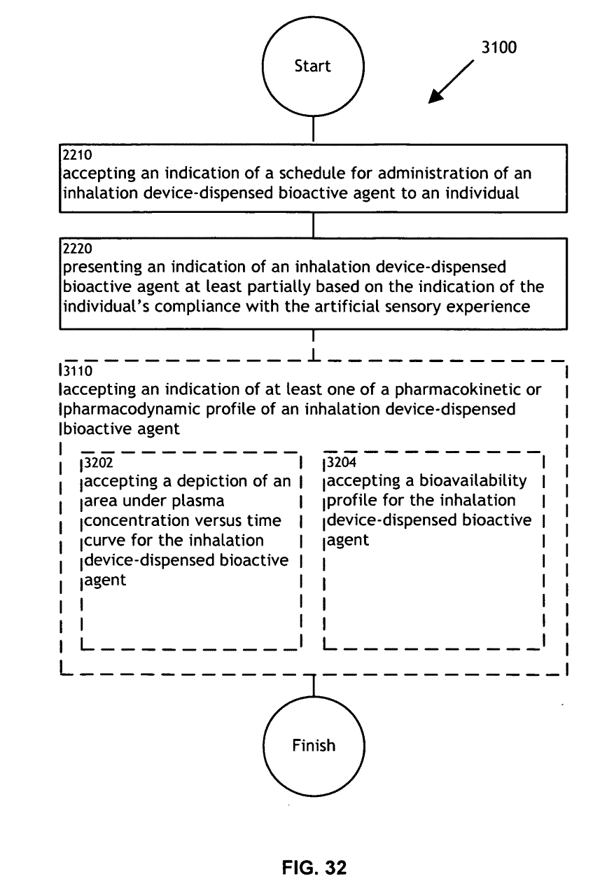

FIG. 32 illustrates alternative embodiments of the example operational flow 3100 of FIG. 31. FIG. 32 illustrates example embodiments where operation 3110 may include at least one additional operation. Additional operations may include operation 3202, and/or operation 3204.

Operation 3202 illustrates accepting a depiction of an area under plasma concentration versus time curve for the inhalation device-dispensed bioactive agent. For example, as shown in FIGS. 18 through 21, area accepter module 2056 may accept a depiction of an area under plasma concentration versus time curve for the inhalation device-dispensed bioactive agent. In one embodiment, area accepter module 2056 may accept a depiction of an area under plasma concentration versus time curve for desflurane. An area under plasma concentration versus time curve may be useful for determining an appropriate time period to administer an artificial sensory experience configured to serve as a distraction and compensate for a reduced bioactive agent efficacy. In some instances, area accepter module 2056 may include a computer processor.

Operation 3204 illustrates accepting a bioavailability profile for the inhalation device-dispensed bioactive agent. For example, as shown in FIGS. 18 through 21, bioavailability profile accepter module 2058 may accept a bioavailability profile for the inhalation device-dispensed bioactive agent. In one embodiment, bioavailability profile accepter module 2058 may accept a bioavailability profile for nitrous oxide. A bioavailability profile may include information describing the fraction of an inhalation device-dispensed bioactive agent dose that may reach systemic circulation in an unchanged state or is therapeutically active. In another embodiment, bioavailability profile accepter module 2058 may accept a bioavailability profile for a albuterol. In some instances, bioavailability profile accepter module 2058 may include a computer processor.

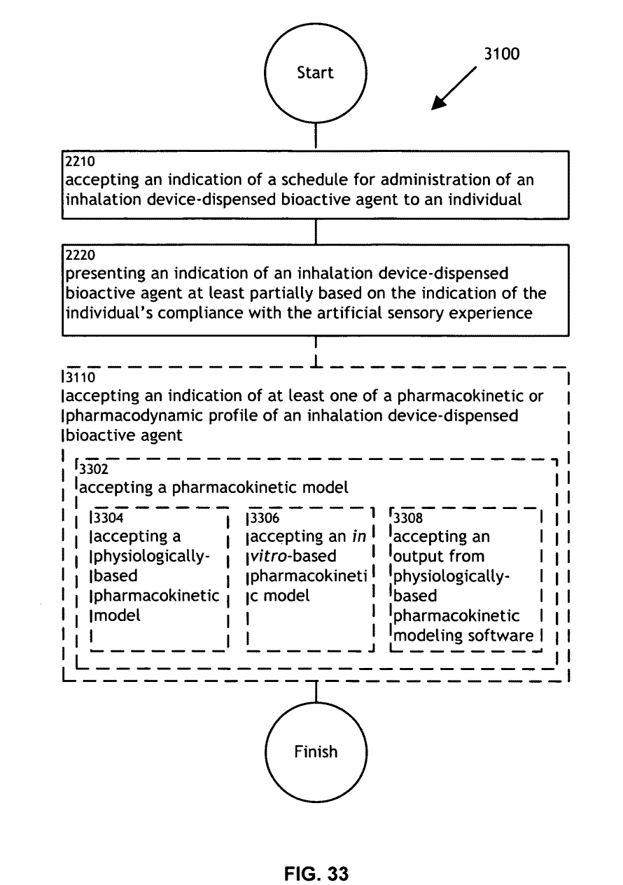

FIG. 33 illustrates alternative embodiments of the example operational flow 3100 of FIG. 31. FIG. 33 illustrates example embodiments where operation 3110 may include at least one additional operation. Additional operations may include operation 3302, operation 3304, operation 3306, and/or operation 3308.

Operation 3302 illustrates accepting a pharmacokinetic model. For example, as shown in FIGS. 18 through 21, model accepter module 2060 may accept a pharmacokinetic model. A pharmacokinetic profile may include information regarding bioactive agent absorption, distribution, metabolism, and/or elimination. In one embodiment, model accepter module 2060 may accept a pharmacokinetic model including a model derived by computer software. A pharmacokinetic model may include, for example, a one-compartment model or a two-compartment model. A one-compartment model may be suitable for an inhalation device-dispensed bioactive agent that rapidly equilibrates in a tissue compartment. A two-compartment model may be suitable for an inhalation device-dispensed bioactive agent that slowly equilibriates in a tissue compartment. Additionally, a pharmacokinetic model may be presented as a data plot, such as a plot of plasma drug concentration versus time. In some instances, model accepter module 2060 may include a computer processor.

Further, operation 3304 illustrates accepting a physiologically-based pharmacokinetic model. For example, as shown in FIGS. 18 through 21, physiologically-based model accepter module 2062 may accept a physiologically-based pharmacokinetic model. In one embodiment, physiologically-based model accepter module 2062 may accept a pharmacokinetic model based on weight and height. A pharmacokinetic model based on weight and height may present pharmacokinetic information based on physiological attributes of an individual or a group of individuals. For example, a physiologically-based pharmacokinetic model may be derived from a clinical trial by using the results only from a group of individuals meeting a specific weight and height requirement. Other physiologic attributes a pharmacokinetic model may be based from may include age, gender, allergy information, and/or racial background. Accepting and/or utilizing a physiologically-based pharmacokinetic model may serve to more accurately determine a pharmacokinetic model for an individual. In some instances, physiologically-based model accepter module 2062 may include a computer processor.

Further, operation 3306 illustrates accepting an in vitro-based pharmacokinetic model. For example, as shown in FIGS. 18 through 21, in vitro-based model accepter module 2064 may accept an in vitro-based pharmacokinetic model. An in vitro-based pharmacokinetic model may include a model derived from a controlled experiment outside of a living organism. One example of an in vitro experiment may include an experiment in a test tube. In one embodiment, in vitro-based model accepter module 2064 may accept an in vitro-based pharmacokinetic model for nitrous oxide developed from a test tube experiment located in a research laboratory. One example of in vitro-based pharmacokinetic modeling software may include DDDPlus, available from Simulations Plus, Inc. Another example of utilizing a physiologic-based simulation model at least partially based on in vitro data may be found in Grass et al., U.S. Pat. No. 6,647,358, which is incorporated herein by reference. In some instances, in vitro-based model accepter module 2064 may include a computer processor.

Further, operation 3308 illustrates accepting an output from physiologically-based pharmacokinetic modeling software. For example, as shown in FIGS. 18 through 21, software output accepter module 2066 may accept an output from physiologically-based pharmacokinetic modeling software. In one embodiment, software output accepter module 2066 may accept an output from physiologically-based pharmacokinetic modeling software. Accepting an output may include, for example, accepting a digital file with pharmacokinetic information and/or accepting printed information, such as a plasma drug concentration versus time graphed depiction. Some examples of physiologically-based pharmacokinetic modeling software may include, for example, acslXtreme, GNU MCSim, PK-Sim, Simcyp, and/or GastroPlus. In some instances, software output accepter module 2066 may include a computer processor.

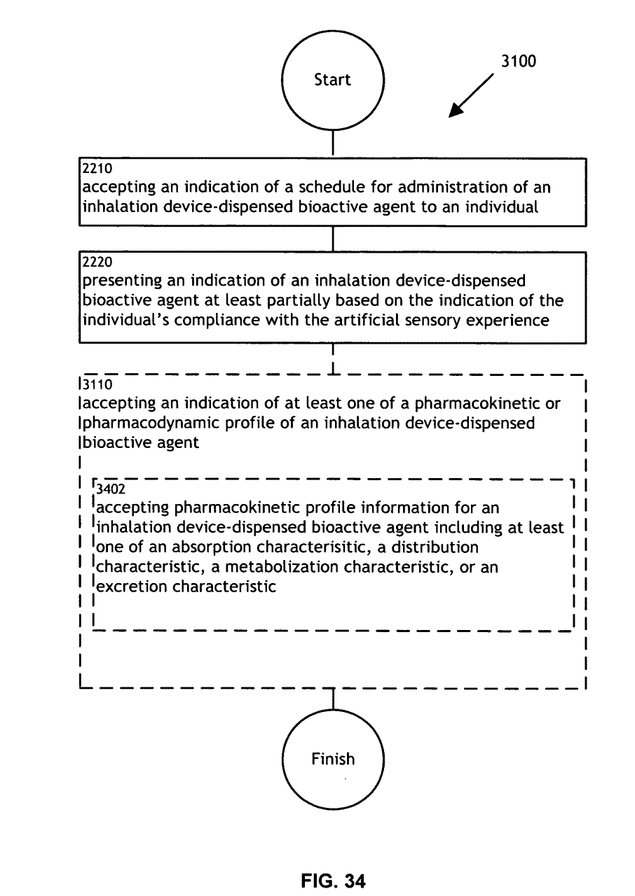

FIG. 34 illustrates alternative embodiments of the example operational flow 3100 of FIG. 31. FIG. 34 illustrates example embodiments where operation 3110 may include at least one additional operation. Additional operations may include operation 3402.

Operation 3402 illustrates accepting pharmacokinetic profile information for an inhalation device-dispensed bioactive agent including at least one of an absorption characteristic, a distribution characteristic, a metabolization characteristic, or an excretion characteristic. For example, as shown in FIGS. 18 through 21, characteristic accepter module 2068 may accept pharmacokinetic profile information for an inhalation device-dispensed bioactive agent including at least one of an absorption characteristic, a distribution characteristic, a metabolization characteristic, or an excretion characteristic. An absorption characteristic may include an inhalation device-dispensed bioactive agent route of administration, the rate of dissolution of an inhalation device-dispensed bioactive agent, and/or ionization of a molecule. In one embodiment, characteristic accepter module 2068 may accept an absorption characteristic including a rate of dissolution for a medication in tablet form having an enteric coating. A distribution characteristic may include permeability between tissues, blood flow and perfusion rate of the tissue, pH parturition, and/or ability of the drug to bind plasma proteins and tissue. In one embodiment, characteristic accepter module 2068 may accept a distribution characteristic for halothane, including a volume of distribution of 3 L/kg of body weight. A metabolization characteristic may include, for example, a metabolic rate and may include and/or be influenced by the presence of a second drug and/or compound. Metabolization may include modification and/or degradation, for example, by an enzyme or enzyme complex. For example, metabolization may include modification of a first drug into a more active version of the first drug and/or a second drug. In one embodiment, characteristic accepter module 2068 may accept a metabolic rate for morphine. An excretion characteristic may include an excretion rate, for example expressed in ng/mL/min. In one embodiment, characteristic accepter module 2068 may accept an excretion characteristic for morphine and/or morphine metabolites, such as 500 ng/mL/min. In some instances, characteristic accepter module 2068 may include a computer processor.

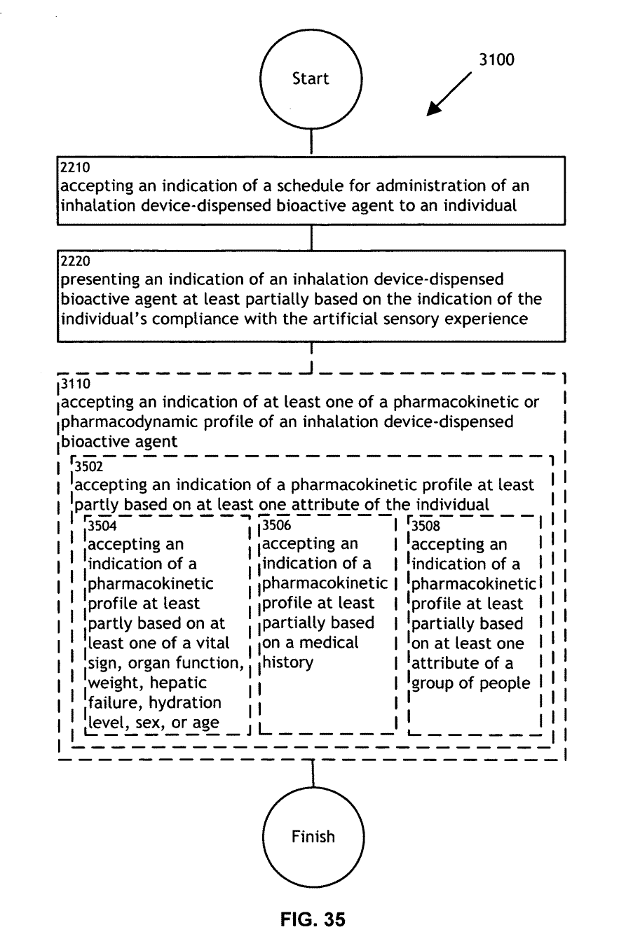

FIG. 35 illustrates alternative embodiments of the example operational flow 3100 of FIG. 31. FIG. 35 illustrates example embodiments where operation 3110 may include at least one additional operation. Additional operations may include operation 3502, operation 3504, operation 3506, and/or operation 3508.

Operation 3502 illustrates accepting an indication of a pharmacokinetic profile at least partly based on at least one attribute of the individual. For example, as shown in FIGS. 18 through 21, individual attribute accepter module 2070 may accept an indication of a pharmacokinetic profile at least partly based on at least one attribute of the individual. In one embodiment, individual attribute accepter module 2070 may accept an indication of a pharmacokinetic profile at least partly based on age and weight of an individual. Accepting an indication of a pharmacokinetic profile at least partly based on an attribute of the individual may serve to more accurately predict the effects of a bioactive agent on the individual by more closely matching the individual's attributes with data from pharmaceutical tests involving people similar to the individual. In some instances, individual attribute accepter module 2070 may include a computer processor.

Further, operation 3504 illustrates accepting an indication of a pharmacokinetic profile at least partly based on at least one of a vital sign, organ function, weight, hepatic failure, hydration level, sex, or age. For example, as shown in FIGS. 18 through 21, individual characteristic accepter module 2072 may accept an indication of a pharmacokinetic profile at least partly based on at least one of a vital sign, organ function, obesity, hepatic failure, hydration level, sex, or age. In one embodiment, individual characteristic accepter module 2072 may accept an indication of a pharmacokinetic profile at least partly based on obesity and age. A vital sign may include a physiological measurement taken in order to assess basic body functions. Some examples of a vital sign may include body temperature, blood pressure, respiratory rate, heart rate, and/or blood oxygen level. Organ function may include the extent to which an organ is performing its expected and/or normal function. Organ function may include organ failure. One example of organ function may include cardiopulmonary function. Other examples of organ function may include, for example, liver function, which may further include decreased renal output, renal insufficiency, hepatic insufficiency, and/or hepatitis. Renal failure may include a situation where at least one kidney fails to properly function. Weight may include obesity, abnormal weight, and/or clinically underweight physiology, for example. Obesity may include a situation where body fat accumulates to a harmful degree. Obesity and/or abnormal weight characteristics may be measured and/or assessed using a body mass index. Hepatic failure may include a situation where the liver is unable to perform its normal metabolic and/or synthetic functions. Hydration level may include the level of body fluids, for example, water. Some effects from hydration level and/or lack of sufficient hydration level may include dehydration, swelling, and/or edema. Dehydration may include the absence and/or removal of water from the body and may negatively affect a bioactive agent effect on an individual. Additionally, sex and age may affect the magnitude of effect the bioactive agent may have on an individual. For example, an elderly person may not be affected as greatly as a young person because of a slower metabolic rate. In some instances, individual characteristic accepter module 2072 may include a computer processor.

Further, operation 3506 illustrates accepting an indication of a pharmacokinetic profile at least partially based on a medical history. For example, as shown in FIGS. 18 through 21, medical history accepter module 2074 may accept an indication of a pharmacokinetic profile at least partially based on a personal medical history. A personal medical history may include a list of previous illnesses, symptoms, medicines, treatments, health risk factors, operations, and/or doctor visits associated with at least one individual. In one embodiment, medical history accepter module 2074 may accept an indication of an individualized pharmacokinetic profile based on previously prescribed medications for an individual. An individualized pharmacokinetic profile may serve to more closely tailor a bioactive agent to the therapeutic needs of the individual. For example, an individualized pharmacokinetic profile may take into account absorption rates for a certain family of inhalation device-dispensed bioactive agents, such as bronchodilators. If the individual reacts to a first bronchodilator in a certain way, the individual may react to a second bronchodilators in a similar way. In some instances, medical history accepter module 2074 may include a computer processor.

Further, operation 3508 illustrates accepting an indication of a pharmacokinetic profile at least partially based on at least one attribute of a group of people. For example, as shown in FIGS. 18 through 21, group attribute accepter module 2076 may accept an indication of a pharmacokinetic profile at least partially based on at least one attribute of a group of people. In one embodiment, group attribute accepter module 2076 may accept an indication of a pharmacokinetic profile based on the weight of a group of people where the group of people may be similar in weight to the individual. Utilizing a pharmacokinetic profile based on a group of people with at least one similar attribute to the individual may serve to better predict how an inhalation device-dispensed bioactive agent may affect the individual. In some instances, group attribute accepter module 2076 may include a computer processor.

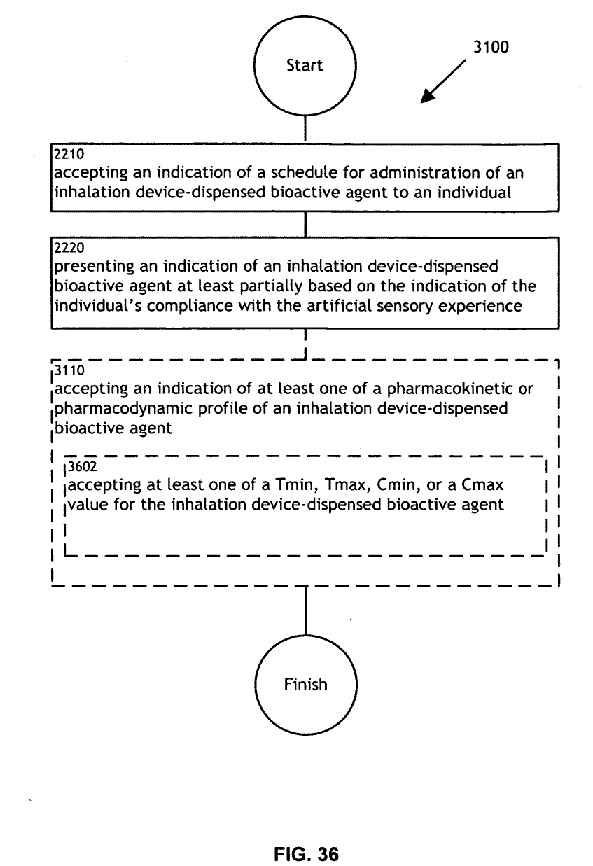

FIG. 36 illustrates alternative embodiments of the example operational flow 3100 of FIG. 31. FIG. 36 illustrates example embodiments where operation 3110 may include at least one additional operation. Additional operations may include operation 3602.

Operation 3602 illustrates accepting at least one of a Tmin, Tmax, Cmin, or a Cmax value for the inhalation device-dispensed bioactive agent. For example, as shown in FIGS. 18 through 21, parameter accepter module 2078 may accept at least one of a Tmin, Tmax, Cmin, or a Cmax value for the bioactive agent. A Tmin value may include a time when an inhalation device-dispensed bioactive agent plasma concentration is at a minimum value. A Tmax value may include a time when an inhalation device-dispensed bioactive agent plasma concentration is at a maximum value. A Cmin value may include a minimum inhalation device-dispensed bioactive agent concentration value. A Cmax value may include a maximum inhalation device-dispensed bioactive agent concentration value. In one embodiment, parameter accepter module 2078 may accept a Tmin and a Tmax value. Accepting a Tmin and a Tmax value for an inhalation device-dispensed bioactive agent during an inhalation device-dispensed bioactive agent regimen may serve to help determine an appropriate time to administer a corresponding artificial sensory experience. An appropriate time to administer a corresponding artificial sensory experience may include a time period when an inhalation device-dispensed bioactive agent is at a low and/or minimum concentration. An artificial sensory experience may compensate for a low and/or minimum inhalation device-dispensed bioactive agent concentration by serving as a distraction. In some instances, parameter accepter module 2078 may include a computer processor.

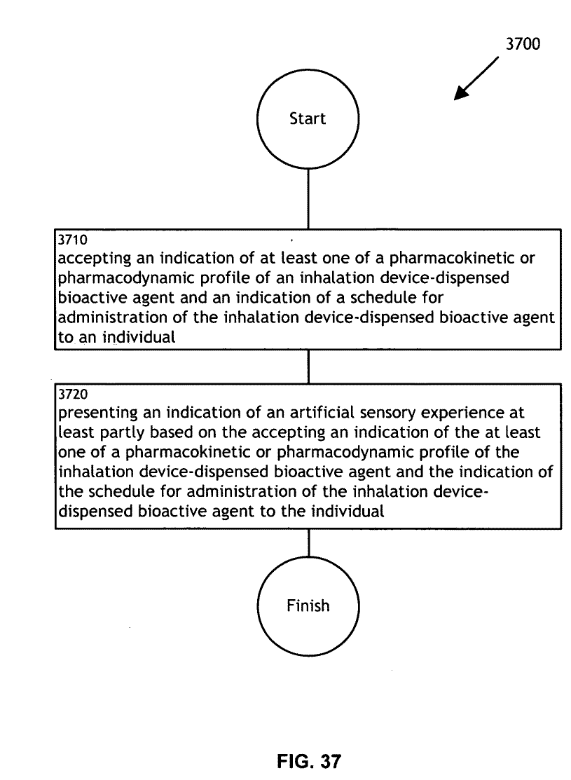

FIG. 37 illustrates an operational flow 3700 representing example operations related to accepting an indication of at least one of a pharmacokinetic or pharmacodynamic profile of an inhalation device-dispensed bioactive agent and an indication of a schedule for administration of the inhalation device-dispensed bioactive agent to an individual and presenting an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In FIG. 37 and in following figures that include various examples of operational flows, discussion and explanation may be provided with respect to the above-described examples of FIGS. 18 through 21, and/or with respect to other examples and contexts. However, it should be understood that the operational flows may be executed in a number of other environments and contexts, and/or in modified versions of FIGS. 18 through 21. Also, although the various operational flows are presented in the sequence(s) illustrated, it should be understood that the various operations may be performed in other orders than those which are illustrated, or may be performed concurrently.

After a start operation, the operational flow 3700 moves to operation 3710. Operation 3710 depicts accepting an indication of at least one of a pharmacokinetic or pharmacodynamic profile of an inhalation device-dispensed bioactive agent and an indication of a schedule for administration of the inhalation device-dispensed bioactive agent to an individual. For example, as shown in FIGS. 18 through 21, accepter module 2002 may accept an indication of at least one of a pharmacokinetic or pharmacodynamic profile of an inhalation device-dispensed bioactive agent and an indication of a schedule for administration of the inhalation device-dispensed bioactive agent to an individual. In one embodiment, accepter module 2002 may accept an indication of a pharmacokinetic profile for an inhaled anesthetic and a schedule for an inhalational administration of the anesthetic to an individual. In this embodiment, the schedule may include specific times and/or methods that the inhalation device-dispensed bioactive agent may be administered. For example, a time schedule may specify that an individual should receive a specific dose of nitrous oxide every two hours. In some instances, accepter module 2002 may include a computer processor and/or a user interface coupled to the computer processor.

Then, operation 3720 depicts presenting an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. For example, as shown in FIGS. 18 through 21, presenter module 2010 may present an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In one embodiment, presenter module 2010 may present an indication of a virtual world configured for distracting an individual at least partly based on accepting an indication of a pharmacokinetic profile for isoflurane and an administration time schedule. In this embodiment, the bioactive administration schedule may be coordinated so that an artificial sensory experience is administered when the isoflurane may be less effective. Coordinating an artificial sensory experience administration schedule may serve to more efficiently distract and/or reduce, for example, an individual's pain during a period of low inhalation device-dispensed bioactive agent concentration in an individual's blood. In some instances, presenter module 2010 may include a computer processor, a display, and/or a printer.

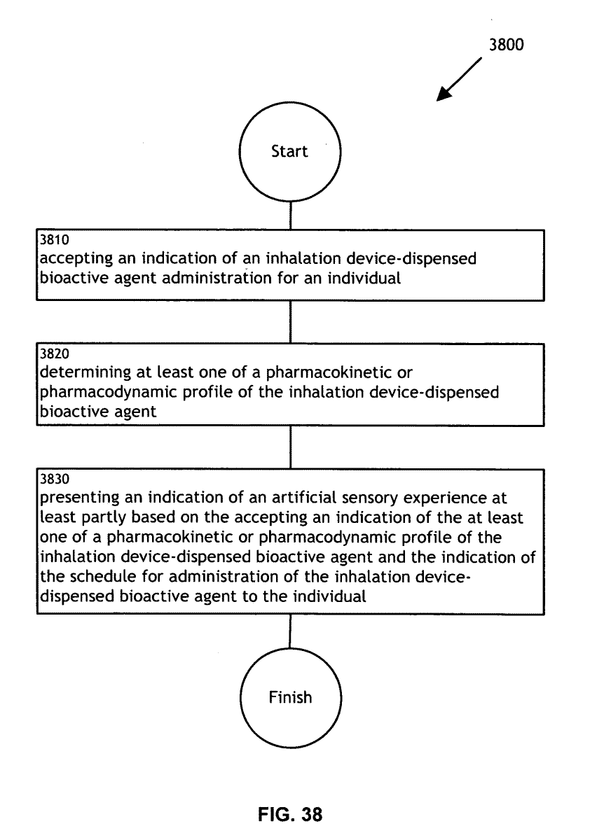

FIG. 38 illustrates an operational flow 3800 representing example operations related to accepting an indication of an inhalation device-dispensed bioactive agent administration for an individual, determining at least one of a pharmacokinetic or pharmacodynamic profile of the inhalation device-dispensed bioactive agent, and presenting an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In FIG. 38 and in following figures that include various examples of operational flows, discussion and explanation may be provided with respect to the above-described examples of FIGS. 18 through 21, and/or with respect to other examples and contexts. However, it should be understood that the operational flows may be executed in a number of other environments and contexts, and/or in modified versions of FIGS. 18 through 21. Also, although the various operational flows are presented in the sequence(s) illustrated, it should be understood that the various operations may be performed in other orders than those which are illustrated, or may be performed concurrently.

After a start operation, the operational flow 3800 moves to a operation 3810. Operation 3810 depicts accepting an indication of an inhalation device-dispensed bioactive agent administration for an individual. For example, as shown in FIGS. 18 through 21, accepter module 2002 may accept an indication of an inhalation device-dispensed bioactive agent administration for an individual. In one embodiment, accepter module 2002 may accept an indication of an administration of an inhaled analgesic, such as nitrous oxide. Accepting an indication of an administration of an inhalation device-dispensed bioactive agent may include, for example, accepting an administration schedule, accepting a delivery method, and/or accepting a bioactive agent type. In some instances, accepter module 2002 may include a computer processor and/or a user interface coupled to the computer processor.

Then, operation 3820 depicts determining at least one of a pharmacokinetic or pharmacodynamic profile of the inhalation device-dispensed bioactive agent. For example, as shown in FIGS. 18 through 21, determiner module 2080 may determine at least one of a pharmacokinetic or pharmacodynamic profile of the inhalation device-dispensed bioactive agent. In one embodiment, determiner module 2080 may determine at least one of a pharmacokinetic profile of an inhaled analgesic, such as nitrous oxide. Determining at least one of a pharmacokinetic or pharmacodynamic profile for an inhalation device-dispensed bioactive agent may include using a medical history, such as a personal medical history including personal medication efficacy and/or individual attributes (gender, weight, height, etc.). Additionally, determiner module 2080 may determine a pharmacokinetic and/or pharmacodynamic profile for an inhalation device-dispensed bioactive agent by comparing an individual's characteristics with predetermined compatable pharmacokinetic and/or pharmacodynamic profiles, for example from a database. In some instances, determiner module 2080 may include a computer processor.

Then, operation 3830 depicts presenting an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. For example, as shown in FIGS. 18 through 21, presenter module 2010 may present an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In one embodiment, presenter module 2010 may present to a computer monitor an indication of a virtual world based on accepting an indication of a pharmacokinetic profile for halothane and an indication of an administration schedule for the halothane. The indication of the artificial sensory experience may be presented, for example, by displaying the indication on a display device, such as a computer monitor, a mobile device, such as a Blackberry device, and/or a printer device, such as a printed piece of paper from a laser printer. In some instances, presenter module 2010 may include a computer processor and/or a display device, such as a computer monitor and/or a printer.

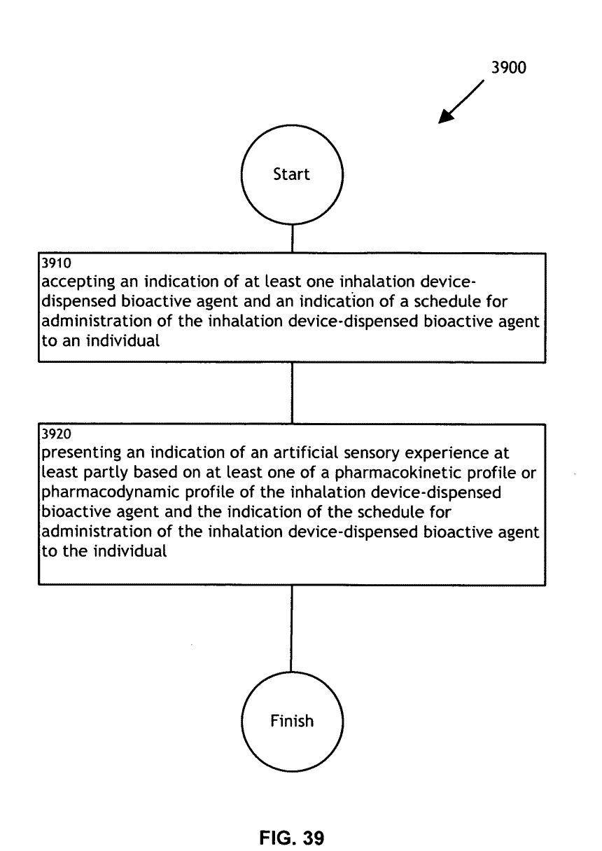

FIG. 39 illustrates an operational flow 3900 representing example operations related to accepting an indication of at least one inhalation device-dispensed bioactive agent and an indication of a schedule for administration of the inhalation device-dispensed bioactive agent to an individual and presenting an indication of an artificial sensory experience at least partly based on at least one of a pharmacokinetic profile or pharmacodynamic profile of the inhalation device-dispensed bioactive agent and the indication of the schedule for administration of the inhalation device-dispensed bioactive agent to the individual. In FIG. 39 and in following figures that include various examples of operational flows, discussion and explanation may be provided with respect to the above-described examples of FIGS. 18 through 21, and/or with respect to other examples and contexts. However, it should be understood that the operational flows may be executed in a number of other environments and contexts, and/or in modified versions of FIGS. 18 through 21. Also, although the various operational flows are presented in the sequence(s) illustrated, it should be understood that the various operations may be performed in other orders than those which are illustrated, or may be performed concurrently.

After a start operation, the operational flow 3900 moves to operation 3910. Operation 3910 depicts accepting an indication of at least one inhalation device-dispensed bioactive agent and an indication of a schedule for administration of the inhalation device-dispensed bioactive agent to an individual. For example, as shown in FIGS. 18 through 21, accepter module 2002 may accept an indication of at least one inhalation device-dispensed bioactive agent and an indication of a schedule for administration of the inhalation device-dispensed bioactive agent to an individual. In one embodiment, accepter module 2002 may accept an indication of an inhaled anaesthetic, such as isoflurane, and accept an indication of an administration of the isoflurane. Accepting an indication of an inhalation device-dispensed bioactive agent may include, for example, accepting an indication of a prescription medication from a health care provider. Additionally, an indication of an inhalation device-dispensed bioactive agent may be accepted from a computer database. Accepting an indication of an inhalation device-dispensed bioactive agent administration schedule may include specific times and/or methods that the inhalation device-dispensed bioactive agent may be administered. Additionally, an inhalation device-dispensed bioactive agent administration schedule may include receiving a dose with or without food, which may be specific food, and/or together with or separate from other drugs. For example, a time schedule may specify that an individual should receive a specific dose of isoflurane every two hours. Accepting an indication of an administration schedule may include accepting a delivery method and/or accepting an inhalation device-dispensed bioactive agent to be administered. In some instances, accepter module 2002 may include a computer processor and/or a user interface coupled to the computer processor.

Then, operation 3920 depicts presenting an indication of an artificial sensory experience at least partly based on at least one of a pharmacokinetic profile or pharmacodynamic profile of the inhalation device-dispensed bioactive agent and the indication of the schedule for administration of the inhalation device-dispensed bioactive agent to the individual. For example, as shown in FIGS. 18 through 21, presenter module 2010 may present an indication of an artificial sensory experience at least partly based on the accepting an indication of the at least one of a pharmacokinetic or pharmacodynamic profile of the inhalation device-dispensed bioactive agent and the indication of the schedule for administration of the inhalation device-dispensed bioactive agent to the individual. In one embodiment, presenter module 2010 may present to a computer monitor an indication of a virtual world based on accepting an indication of a pharmacokinetic profile for albuterol and an indication of an administration schedule for the albuterol. The indication of the artificial sensory experience may be presented, for example, by displaying the indication on a display device, such as a computer monitor, a mobile device, such as a Blackberry device, and/or a printer device, such as a printed piece of paper from a laser printer. In some instances, presenter module 2010 may include a computer processor and/or a display device, such as a computer monitor and/or a printer.

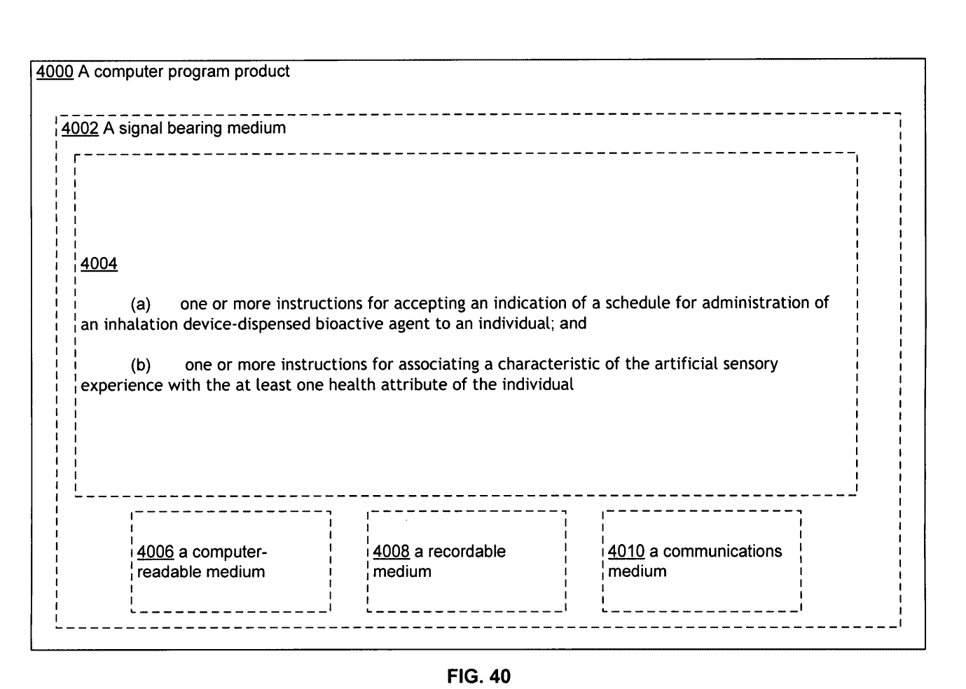

FIG. 40 illustrates a partial view of an example computer program product 4000 that includes a computer program 4004 for executing a computer process on a computing device. An embodiment of the example computer program product 4000 is provided using a signal-bearing medium 4002, and may include one or more instructions for accepting an indication of a schedule for administration of an inhalation device-dispensed bioactive agent to an individual and one or more instructions for presenting an indication of an artificial sensory experience at least partly based on the accepting an indication of the schedule for administration of the inhalation device-dispensed bioactive agent to the individual. The one or more instructions may be, for example, computer executable and/or logic-implemented instructions. In one implementation, the signal-bearing medium 4002 may include a computer-readable medium 4006. In one implementation, the signal bearing medium 4002 may include a recordable medium 4008. In one implementation, the signal bearing medium 4002 may include a communications medium 4010.

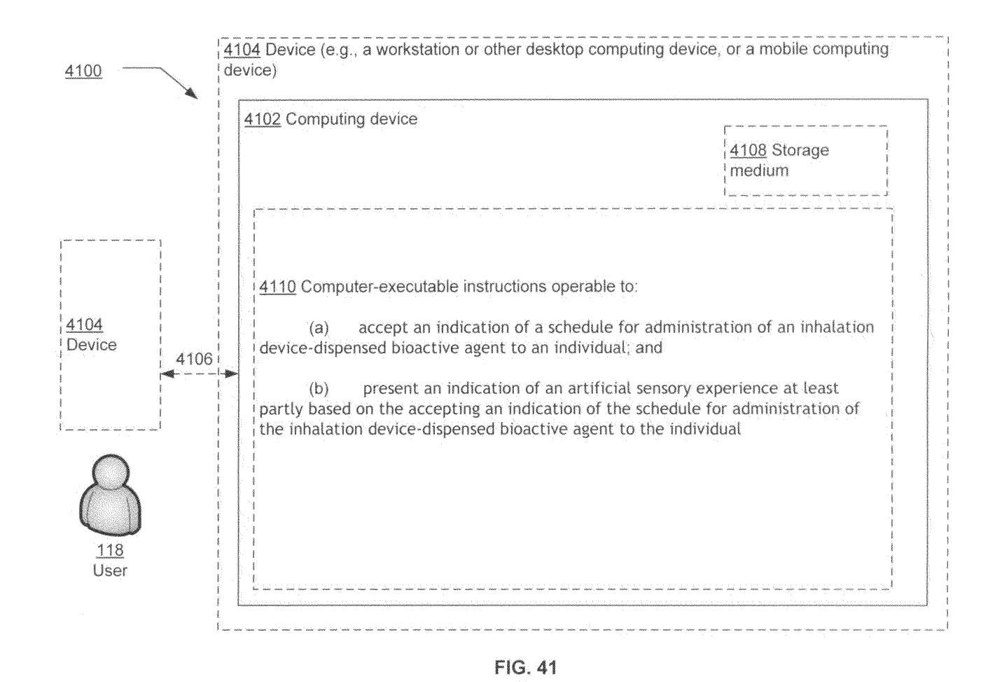

FIG. 41 illustrates an example system 4100 in which embodiments may be implemented. The system 4100 includes a computing system environment. The system 4100 also illustrates the user 118 using a device 4104, which is optionally shown as being in communication with a computing device 4102 by way of an optional coupling 4106. The optional coupling 4106 may represent a local, wide-area, or peer-to-peer network, or may represent a bus that is internal to a computing device (e.g., in example embodiments in which the computing device 4102 is contained in whole or in part within the device 4104). A storage medium 4108 may be any computer storage media.

The computing device 4102 includes computer-executable instructions 4110 that when executed on the computing device 4102 cause the computing device 4102 to accept an indication of a schedule for administration of an inhalation device-dispensed bioactive agent to an individual and present an indication of an artificial sensory experience at least partly based on the accepting an indication of the schedule for administration of the inhalation device-dispensed bioactive agent to the individual. As referenced above and as shown in FIG. 41, in some examples, the computing device 4102 may optionally be contained in whole or in part within the device 4104.

In FIG. 41, then, the system 4100 includes at least one computing device (e.g., 4102 and/or 4104). The computer-executable instructions 4110 may be executed on one or more of the at least one computing device. For example, the computing device 4102 may implement the computer-executable instructions 4110 and output a result to (and/or receive data from) the computing device 4104. Since the computing device 4102 may be wholly or partially contained within the computing device 4104, the device 4104 also may be said to execute some or all of the computer-executable instructions 4110, in order to be caused to perform or implement, for example, various ones of the techniques described herein, or other techniques.

The device 4104 may include, for example, a portable computing device, workstation, or desktop computing device. In another example embodiment, the computing device 4102 is operable to communicate with the device 4104 associated with the user 118 to receive information about the input from the user 118 for performing data access and data processing and presenting an output of the user-health test function at least partly based on the user data.

Although a user 118 is shown/described herein as a single illustrated figure, those skilled in the art will appreciate that a user 118 may be representative of a human user, a robotic user (e.g., computational entity), and/or substantially any combination thereof (e.g., a user may be assisted by one or more robotic agents). In addition, a user 118, as set forth herein, although shown as a single entity may in fact be composed of two or more entities. Those skilled in the art will appreciate that, in general, the same may be said of “sender” and/or other entity-oriented terms as such terms are used herein.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation utilized herein (e.g., beginning with a presentation of a flowchart(s) presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in tight of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the art will recognize that the various embodiments described herein can be implemented, individually and/or collectively, by various types of electromechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electromechanical systems include but are not limited to a variety of consumer electronics systems, medical devices, as well as other systems such as motorized transport systems, factory automation systems, security systems, and/or communication/computing systems. Those skilled in the art will recognize that electromechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory.

Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although user 118 is shown/described herein as a single illustrated figure, those skilled in the art will appreciate that user 118 may be representative of a human user, a robotic user (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art wilt recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A computer-implemented method, comprising:
accepting an indication of a schedule for administration of an inhalation device-dispensed bioactive agent to an individual; and
presenting an indication of an artificial sensory experience at least partly based on the accepting an indication of the schedule for administration of the inhalation device-dispensed bioactive agent to the individual.

2-43. (canceled)

44. A system, comprising:
means for accepting an indication of a schedule for administration of an inhalation device-dispensed bioactive agent to an individual; and
means for presenting an indication of an artificial sensory experience at least partly based on the accepting an indication of the schedule for administration of the inhalation device-dispensed bioactive agent to the individual.

45. The system of claim 44, wherein the means for accepting an indication of a schedule for administration of an inhalation device-dispensed bioactive agent to an individual comprises:
means for accepting an inhalation device-dispensed bioactive agent time schedule.

46. The system of claim 44, wherein the means for accepting an indication of a schedule for administration of an inhalation device-dispensed bioactive agent to an individual comprises:
means for accepting at least one of an inhalation device-dispensed bioactive agent delivery type or inhalation device-dispensed bioactive agent delivery dosage administration schedule.

47. The system of claim 44, wherein the means for accepting an indication of a schedule for administration of an inhalation device-dispensed bioactive agent to an individual comprises:
means for accepting an inhalation device-dispensed bioactive agent loading dose.

48. The system of claim 44, wherein the means for presenting an indication of an artificial sensory experience at least partly based on the accepting an indication of the schedule for administration of the inhalation device-dispensed bioactive agent to the individual comprises:
means for utilizing an algorithm for recommending at least one artificial sensory experience.

49-51. (canceled)

52. The system of claim 44, wherein the means for presenting an indication of an artificial sensory experience at least partly based on the accepting an indication of the schedule for administration of the inhalation device-dispensed bioactive agent to the individual comprises:
means for presenting an indication of an artificial sensory experience at least partly based on a personal medical history.

53. The system of claim 44, wherein the means for presenting an indication of an artificial sensory experience at least partly based on the accepting an indication of the schedule for administration of the inhalation device-dispensed bioactive agent to the individual comprises:
means for presenting an indication of an artificial sensory experience at least partly based on experimental data.

54. The system of claim 44, wherein the means for presenting an indication of an artificial sensory experience at least partly based on the accepting an indication of the schedule for administration of the inhalation device-dispensed bioactive agent to the individual comprises:
means for presenting an indication of an artificial sensory experience at least partly based on a medical reference tool.

55. The system of claim 44, wherein the means for presenting an indication of an artificial sensory experience at least partly based on the accepting an indication of the schedule for administration of the inhalation device-dispensed bioactive agent to the individual comprises:
means for presenting the indication to at least one output device.

56-57. (canceled)

58. The system of claim 44, wherein the means for presenting an indication of an artificial sensory experience at least partly based on the accepting an indication of the schedule for administration of the inhalation device-dispensed bioactive agent to the individual comprises:
means for presenting the indication to a third party.

59. The system of claim 58, wherein the means for presenting the indication to a third party comprises:
means for presenting the indication to a health care provider.

60. The system of claim 58, wherein the means for presenting the indication to a third party comprises:
means for selectively presenting the indication only to the individual.

61. The system of claim 44, wherein the means for presenting an indication of an artificial sensory experience at least partly based on the accepting an indication of the schedule for administration of the inhalation device-dispensed bioactive agent to the individual comprises:
means for presenting an indication of a prescribed artificial sensory experience.

62. The system of claim 61, wherein the means for presenting an indication of a prescribed artificial sensory experience comprises:
means for presenting an indication of at least one effect of the prescribed artificial sensory experience.

63-64. (canceled)

65. The system of claim 61, wherein the means for presenting an indication of a prescribed artificial sensory experience comprises:
means for presenting an indication of at least one time period of an expected change in inhalation device-dispensed bioactive agent effectiveness.

66. The system of claim 61, wherein the means for presenting an indication of a prescribed artificial sensory experience comprises:
means for presenting an indication of at least one time period of an expected change in inhalation device-dispensed bioactive agent blood concentration.

67. The system of claim 61, wherein the means for presenting an indication of a prescribed artificial sensory experience comprises:
means for recommending an artificial sensory experience administration schedule.

68. The system of claim 44, wherein the means for accepting an indication of a schedule for administration of an inhalation device-dispensed bioactive agent to an individual and means for presenting an indication of an artificial sensory experience at least partly based on the accepting an indication of the schedule for administration of the inhalation device-dispensed bioactive agent to the individual comprises:
means for accepting the pharmacokinetic profile of an inhaled bronchodilator, accepting a prescribed administration schedule of the inhaled bronchodilator to an individual, and presenting a prescription for engagement of the individual with a virtual world experience at times when the plasma concentration of the inhaled bronchodilator is expected to be below a substantially effective level based on the pharmacokinetic profile of the inhaled bronchodilator and the prescribed administration schedule of the inhaled bronchodilator.

69. The system of claim 44, further comprising:
means for accepting an indication of at least one of a pharmacokinetic or pharmacodynamic profile of an inhalation device-dispensed bioactive agent.

70. The system of claim 69, wherein the means for accepting an indication of at least one of a pharmacokinetic or pharmacodynamic profile of an inhalation device-dispensed bioactive agent comprises:
means for accepting an indication of a time versus concentration representation for an inhalation device-dispensed bioactive agent.

71. The system of claim 69, wherein the means for accepting an indication of at least one of a pharmacokinetic or pharmacodynamic profile of an inhalation device-dispensed bioactive agent comprises:
means for accepting an indication of a dose versus response representation for the inhalation device-dispensed bioactive agent.

72. The system of claim 69, wherein the means for accepting an indication of at least one of a pharmacokinetic or pharmacodynamic profile of an inhalation device-dispensed bioactive agent comprises:
means for accepting a depiction of an area under plasma concentration versus time curve for the inhalation device-dispensed bioactive agent.

73. The system of claim 69, wherein the means for accepting an indication of at least one of a pharmacokinetic or pharmacodynamic profile of an inhalation device-dispensed bioactive agent comprises:
means for accepting a bioavailability profile for the inhalation device-dispensed bioactive agent.

74. The system of claim 69, wherein the means for accepting an indication of at least one of a pharmacokinetic or pharmacodynamic profile of an inhalation device-dispensed bioactive agent comprises:
means for accepting a pharmacokinetic model.

75-77. (canceled)

78. The system of claim 69, wherein the means for accepting an indication of at least one of a pharmacokinetic or pharmacodynamic profile of an inhalation device-dispensed bioactive agent comprises:
means for accepting pharmacokinetic profile information for an inhalation device-dispensed bioactive agent including at least one of an absorption characteristic, a distribution characteristic, a metabolization characteristic, or an excretion characteristic.

79. The system of claim 69, wherein the means for accepting an indication of at least one of a pharmacokinetic or pharmacodynamic profile of an inhalation device-dispensed bioactive agent comprises:
means for accepting an indication of a pharmacokinetic profile at least partly based on at least one attribute of the individual.

80. The system of claim 79, wherein the means for accepting an indication of a pharmacokinetic profile at least partly based on at least one attribute of the individual comprises:
means for accepting an indication of a pharmacokinetic profile at least partly based on at least one of a vital sign, organ function, weight, hepatic failure, hydration level, sex, or age.

81-82. (canceled)

83. The system of claim 69, wherein the means for accepting an indication of at least one of a pharmacokinetic or pharmacodynamic profile of an inhalation device-dispensed bioactive agent comprises:
means for accepting at least one of a Tmin, Tmax, Cmin, or a Cmax value for the inhalation device-dispensed bioactive agent.

84-86. (canceled)

87. A system, comprising:
circuitry for accepting an indication of a schedule for administration of an inhalation device-dispensed bioactive agent to an individual; and
circuitry for presenting an indication of an artificial sensory experience at least partly based on the accepting an indication of the schedule for administration of the inhalation device-dispensed bioactive agent to the individual.

88. A computer program product comprising:
a signal-bearing medium bearing
one or more instructions for accepting an indication of a schedule for administration of an inhalation device-dispensed bioactive agent to an individual; and
one or more instructions for presenting an indication of an artificial sensory experience at least partly based on the accepting an indication of the schedule for administration of the inhalation device-dispensed bioactive agent to the individual.

89. The computer program product of claim 88, wherein the signal-bearing medium includes a computer-readable medium.

90. The computer program product of claim 88, wherein the signal-bearing medium includes a recordable medium.

91. The computer program product of claim 88, wherein the signal-bearing medium includes a communications medium.

92. A system comprising:
a computing device; and
instructions that when executed on the computing device cause the computing device to
accept an indication of a schedule for administration of an inhalation device-dispensed bioactive agent to an individual; and
present an indication of an artificial sensory experience at least partly based on the accepting an indication of the schedule for administration of the inhalation device-dispensed bioactive agent to the individual.

93. The system of claim 92 wherein the computing device comprises:
one or more of a personal digital assistant (PDA), a personal entertainment device, a mobile phone, a laptop computer, a tablet personal computer, a networked computer, a computing system comprised of a cluster of processors, a computing system comprised of a cluster of servers, a workstation computer, and/or a desktop computer.

94. The system of claim 92, wherein the computing device is operable to accept the at least one attribute of the at least one individual and present the indication of the at least one prescription medication and the at least one artificial sensory experience from at least one memory.
